(19) United States
(12) Patent Application Publication
(10) Pub. No.: US 2025/0282764 A1
(43) Pub. Date: Sep. 11, 2025

(54) IMIDAZOLE HETEROCYCLE BASED COMPOUNDS AND METHODS OF TREATING CANCER

(71) Applicant: King Abdulaziz University, Jeddah (SA)

(72) Inventors: Sherin BAKHASHAB, Jeddah (SA); Salha ALSHAMRANI, Jeddah (SA); Farid AHMED, Jeddah (SA); Muhammad Imran NASEER, Jeddah (SA); Abdelsattar M. OMAR, Jeddah (SA); Afaf A. EL-MALAH, Jeddah (SA); Moustafa E. EL-ARABY, Richmond, VA (US); Reem ALSOLAMI, Jeddah (SA); Yosra A. MUHAMMAD, Jeddah (SA); Peter Natesan PUSHPARAJ, Jeddah (SA)

(73) Assignee: King Abdulaziz University, Jeddah (SA)

(21) Appl. No.: 18/601,388

(22) Filed: Mar. 11, 2024

(51) Int. Cl. C07D 405/06; A61K 31/4178; A61P 35/00; A61P 35/02

(52) U.S. Cl. CPC C07D 405/06; A61K 31/4178; A61P 35/00; A61P 35/02

(57) ABSTRACT

A compound, having a following general formula (I):

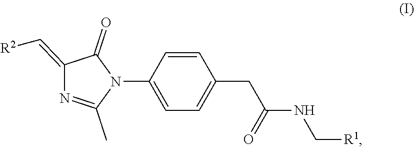

a salt thereof, a solvate thereof, a tautomer thereof, a stereoisomer thereof, or a mixture thereof, where R¹ is selected from the group consisting of an optionally substituted alkyl, an optionally substituted cycloalkyl, an optionally substituted arylalkyl, and an optionally substituted aryl, and R² is an optionally substituted heterocycle.

### STATEMENT OF ACKNOWLEDGEMENT

The support from the Ministry of Education and King Abdulaziz University, Jeddah, Saudi Arabia for this research through a grant (IFPNC-012-141-2020) is gratefully acknowledged.

### BACKGROUND OF THE INVENTION

### Technical Field

The present disclosure relates to imidazole heterocycle-based compounds, a pharmaceutical composition containing the compounds, and a method for treating cancer with the compounds.

### Description of the Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

Colorectal cancer (CRC) is the third most common cancer and the second leading cause of mortality. Prognostic biomarkers help in determining the benefits of adjuvant chemotherapy. Despite the increasingly detailed molecular mapping of CRC, only a few markers have shown promise in clinical practice in terms of their ability to predict the disease course, such as amplification of oncogenes, including epidermal growth factor receptor (EGFR), ERBB2, CCND1, MET, and MYC, mutations within KRAS, BRAF proto-oncogenes and deletions of tumor suppressors, including, tumor protein 53 (TP53) and SMAD4.

5-Fluorouracil (5-FU) is one of the most widely used antimetabolite drugs for the first-line therapy of CRC. The standard treatment for locally advanced CRC cancer is surgical resection followed by adjuvant treatment with 5-FU and leucovorin. 5-FU is a pyrimidine analog and inhibitor of thymidylate synthase, which inhibits DNA synthesis and causes cell death. Several studies have shown chemoresistance to 5-FU for CRC patients with loss of SMAD4 expression. In addition, molecular-targeted drugs, including EGFR antagonists and angiogenesis inhibitors, have been introduced for the management of metastatic CRC. For example, cetuximab, a monoclonal antibody against the extracellular binding domain of EGFR, the first-line treatment with cetuximab plus FOLFIRI reduced the risk of progression of metastatic CRC when compared with FOLFIRI alone; however, the benefit of cetuximab was limited in patients with KRAS wild-type tumors. The addition of cetuximab to FOLFIRI has been shown to increase the median progression-free survival of patients with previously untreated RAS wild-type metastatic disease by approximately three months. However, most patients either have a poor initial response to treatment or develop secondary resistance to standard therapies. Therefore, there exists a need to identify new therapeutic compounds with anti-cancer activities.

Acute myeloid leukemia (AML) is a cancer of the blood forming myeloid cells. It is an aggressive cancer which requires appropriate diagnosis and immediate intervention. Conventional chemotherapy, allogeneic stem cell transplantation, and in some cases targeted therapies are the current standard of care for AML. A major concern in AML therapy is the poor outcome of the disease. More than 60% of AML patients treated with standard chemotherapy across all age groups eventually relapse. The 5-year survival rates following standard intensive chemotherapy is 30-35% in patients up to the age of 60 years, however, in patients over 60 years the 5-year survival rates are <10-15%. Treatment related mortality and multidrug resistance are believed to be the main reasons for the failure of current AML treatments. Using a combination of drugs that simultaneously inhibits multiple oncogenic targets is an alternative strategy for targeting AML. However, combination therapies are often hindered by complex drug-drug interactions and unclear pharmacokinetic profiles. In contrast, single agents that target multiple members of the oncogenic signaling pathway are expected to overcome the drawbacks associated with use of single-target inhibitors and drug combinations.

In a previous study, compound KIM-161, possessing a 5-arylidene-1,4-dihydro-4H-imidazol-5-one scaffold, was discovered as an inhibitor of proliferation of a variety of cancer cell lines (Khayat, M. T.; Omar, A. M.; Ahmed, F.; Khan, M. I.; Ibrahim, S. M.; Muhammad, Y. A.; Malebari, A. M.; Neamatallah, T.; El-Araby, M. E. Insights on Cancer Cell Inhibition, Subcellular Activities, and Kinase Profile of Phenylacetamides Pending 1H-Imidazol-5-One Variants. Front. Pharmacol. 2022, 12, referred to throughout as Khayat, et. al., and is incorporated herein by reference in its entirety). KIM-161 (depicted in FIG. 1) had good cytotoxic activity against solid tumor cells: PC3 prostate cancer (IC₅₀ 2.135 μM), MCF7 breast cancer (IC₅₀ 1.141 μM), and HCT116 CRC cells (IC₅₀ 0.294 μM). The compound's antiproliferative effect on NB4, HL60, and KG1a leukemia cell lines was higher with IC₅₀ values of 0.275-0.893 μM. Furthermore, a concentration of 1 μM of KIM-161 caused cell cycle arrest at pre-G and G2/M phases of cell cycle, and it promoted apoptosis through activation of caspase-3. According to the in silico physicochemical profiling, KIM-161 demonstrated acceptable properties such as maximum aqueous solubility (19.9 μM), microsomal stability, as well as plasma stability. Additionally, the compound was not a substrate for the major CYP450 metabolizing enzymes (3A4, 2D6, and 2C9). Nonetheless, the presence of 4-methoxyphenyl as the 5-arylidene moiety poses a challenge in solubilizing the compound in the different dosage forms, especially for IV administration, for in vivo studies.

In view of the forgoing, it is one object of the present disclosure to design a derivative of KIM-161 with improved aqueous solubility to facilitate compound administration and a method for treating cancer with the compounds.

### BRIEF SUMMARY OF THE DISCLOSURE

A compound, having a following general formula (I):

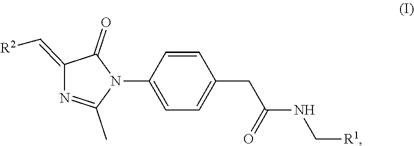

a salt thereof, a solvate thereof, a tautomer thereof, a stereoisomer thereof, or a mixture thereof;
wherein:
R¹ is selected from the group consisting of an optionally substituted alkyl, an optionally substituted cycloalkyl, an optionally substituted arylalkyl, and an optionally substituted aryl, and
R² is an optionally substituted heterocycle.

In some embodiments, R¹ is an optionally substituted aryl.

In some embodiments, R¹ is a phenyl.

In some embodiments, R² is an optionally substituted heteroaryl.

In some embodiments, R² is an optionally substituted heteroaryl having a 5-membered ring.

In some embodiments, R² is an alkyl substituted furyl, imidazolyl, thiazolyl, thienyl, oxazolyl, isoxazolyl, triazolyl, pyrazolyl, isothiazolyl, or pyrroyl.

In some embodiments, R² is an optionally substituted heteroaryl having a 6-membered ring.

In some embodiments, R² is an alkyl substituted pyridyl, pyrimidinyl, pyrazinyl, pyridazinyl, or triazinyl.

In some embodiments, the compound has the following formula (Id):

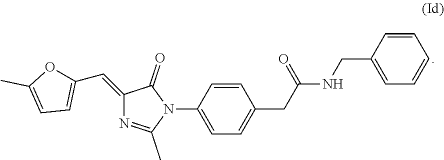 [SMILES: CC1=N/C(=C\c2ccc(C)o2)C(=O)N1c1ccc(CC(=O)NCc2ccccc2)cc1]

In some embodiments, the compound has a maximum kinetic aqueous solubility of greater than 75 μM.

The present disclosure also provides a pharmaceutical composition, including the compound of formula (I) and a pharmaceutically acceptable carrier and/or excipient.

In some embodiments, the pharmaceutical composition includes 0.1-10 wt. % of the compound of formula (I) relative to a total weight of the pharmaceutical composition.

In some embodiments, the pharmaceutically acceptable carrier and/or excipient is at least one selected from the group consisting of a buffer, an inorganic salt, a fatty acid, a vegetable oil, a synthetic fatty ester, a surfactant, and a polymer.

In some embodiments, pharmaceutical composition includes the compound having the following formula (Id):

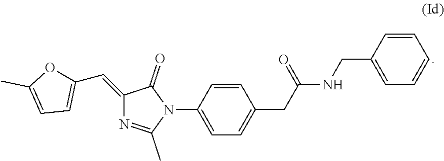 [SMILES: CC1=N/C(=C\c2ccc(C)o2)C(=O)N1c1ccc(CC(=O)NCc2ccccc2)cc1]

The present disclosure also provides a method for treating cancer in a subject, including administering to the subject a therapeutically effective amount of the compound of formula (I).

In some embodiments, the cancer is colon cancer or leukemia.

The present disclosure also provides a method for decreasing an amount of colorectal cancer cells, including contacting the compound of formula (I) with the colorectal cancer cells.

In some embodiments, the compound of formula (I) has an IC₅₀ value of 1-2 μM for the colorectal cancer cells.

The present disclosure also provides a method for decreasing an amount of leukemia cancer cells, including contacting the compound of formula (I) with the leukemia cancer cells.

In some embodiments, the compound of formula (I) has an IC₅₀ value of 0.1-1 μM for the leukemia cancer cells.

The foregoing paragraphs have been provided by way of general introduction and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 illustrates a development of the compound (Id) of the present disclosure from compounds disclosed in Khayat, et. al, specifically KIM-161.

FIG. 2 illustrates a synthetic scheme for the synthesis of the compound (Id) of the present disclosure, according to certain embodiments.

FIG. 3A is a graph illustrating a dose-response curve of compound (Id) on colorectal cancer cell lines HCT-116, DLD1, and HT-29, according to certain embodiments.

FIG. 3B is a graph illustrating a dose-response curve of KIM-161 on colorectal cancer cell lines HCT-116, DLD1, and HT-29, according to certain embodiments.

FIG. 4A are dot plots from flow cytometry analysis of surface Annexin V expression and 7AAD staining depicting the effect of different concentrations of compound (Id) on apoptosis in DLD1, HCT-116, and HT29 cells, according to certain embodiments.

FIG. 4B is a bar graph illustrating an apoptosis percentage after treatment with different concentrations (IC₅₀ and 2× IC₅₀) of compound (Id) on colorectal cancer cell line DLD1, according to certain embodiments.

FIG. 4C is a bar graph illustrating an apoptosis percentage after treatment with different concentrations (IC₅₀ and 2× IC₅₀) of compound (Id) on colorectal cancer cell line HCT-116, according to certain embodiments.

FIG. 4D is a bar graph illustrating an apoptosis percentage after treatment with different concentrations (IC₅₀ and 2× IC₅₀) of compound (Id) on colorectal cancer cell line HT-29, according to certain embodiments.

FIG. 5A depicts a dose-response curve of KIM-161 on leukemia cell lines HL60, MV4-11, and K562, according to certain embodiments.

FIG. 5B depicts a dose-response curve of KIM-161 on leukemia cell lines Kasumi-1, NB4, and KG1a, according to certain embodiments.

FIG. 5C depicts a dose-response curve of compound (Id) on leukemia cell lines HL60, MV4-11, and K562, according to certain embodiments.

FIG. 5D depicts a dose-response curve of compound (Id) on leukemia cell lines Kasumi-1, NB4, and KG1a, according to certain embodiments.

FIG. 6A are dot plots from flow cytometry analysis of surface Annexin V expression and 7AAD staining depicting the effect of different concentrations of compound (Id) on apoptosis in HL60, MV4-11, and K562 cells, according to certain embodiments.

FIG. 6B are dot plots from flow cytometry analysis of surface Annexin V expression and 7AAD staining depicting the effect of different concentrations of compound (Id) on apoptosis in Kasumi-1, NB4, and KG1a cells, according to certain embodiments.

FIG. 7 are bar graphs illustrating an apoptosis percentage after treatment with compound (Id) on leukemia cancer cell lines HL60, MV4-11, K562, Kasumi-1, NB4, and KG1a cells, according to certain embodiments.

### DETAILED DESCRIPTION OF THE EMBODIMENTS

Embodiments of the present disclosure will now be described more fully hereinafter with reference to the accompanying drawings, in which some, but not all embodiments of the disclosure are shown.

### Definitions

As used herein, the terms “compound”, “complex”, and “product” are used interchangeably, and are intended to refer to a chemical entity, whether in the solid, liquid or gaseous phase, and whether in a crude mixture or purified and isolated.

Throughout the specification and the appended claims, a given chemical formula or name shall encompass all stereo and optical isomers and racemates thereof where such isomers exist. Unless otherwise indicated, all chiral (enantiomeric and diastereomeric) and racemic forms are within the scope of the disclosure. Many geometric isomers of C═C double bonds, C═N double bonds, ring systems, and the like can also be present in the compounds, and all such stable isomers are contemplated in the present disclosure. Cis- and trans- (or E- and Z-) geometric isomers of the compounds of the present disclosure are described and may be isolated as a mixture of isomers or as separated isomeric forms. The present compounds can be isolated in optically active or racemic forms. Optically active forms may be prepared by resolution of racemic forms or by synthesis from optically active starting materials. All processes used to prepare compounds of the present disclosure and intermediates made therein are considered to be part of the present disclosure. When enantiomeric or diastereomeric products are prepared, they may be separated by conventional methods, for example, by chromatography, fractional crystallization, or through the use of a chiral agent. Depending on the process conditions the end products of the present disclosure are obtained either in free (neutral) or salt form. Both the free form and the salts of these end products are within the scope of the disclosure. If so desired, one form of a compound may be converted into another form. A free base or acid may be converted into a salt; a salt may be converted into the free compound or another salt; a mixture of isomeric compounds of the present disclosure may be separated into the individual isomers. Compounds of the present disclosure, free form and salts thereof, may exist in multiple tautomeric forms, in which hydrogen atoms are transposed to other parts of the molecules and the chemical bonds between the atoms of the molecules are consequently rearranged. It should be understood that all tautomeric forms, insofar as they may exist, are included within the disclosure. Further, a given chemical formula or name shall encompass all conformers, rotamers, or conformational isomers thereof where such isomers exist. Different conformations can have different energies, can usually interconvert, and are very rarely isolatable. There are some molecules that can be isolated in several conformations. For example, atropisomers are isomers resulting from hindered rotation about single bonds where the steric strain barrier to rotation is high enough to allow for the isolation of the conformers. It should be understood that all conformers, rotamers, or conformational isomer forms, insofar as they may exist, are included within the present disclosure.

As used herein, the term “solvate” refers to a physical association of a compound of this disclosure with one or more solvent molecules, whether organic or inorganic. This physical association includes hydrogen bonding. In certain instances, the solvate will be capable of isolation, for example when one or more solvent molecules are incorporated in the crystal lattice of the crystalline solid. The solvent molecules in the solvate may be present in a regular arrangement and/or a non-ordered arrangement. The solvate may comprise either a stoichiometric or nonstoichiometric amount of the solvent molecules. Solvate encompasses both solution phase and isolable solvates. Exemplary solvents include, but are not limited to, water, methanol, ethanol, n-propanol, isopropanol, n-butanol, isobutanol, tert-butanol, ethyl acetate and other lower alkanols, glycerine, acetone, dichloromethane (DCM), dimethyl sulfoxide (DMSO), dimethyl acetate (DMA), dimethylformamide (DMF), isopropyl ether, acetonitrile, toluene, N-methylpyrrolidone (NMP), tetrahydrofuran (THF), tetrahydropyran, other cyclic mono-, di- and tri-ethers, polyalkylene glycols (e.g., polyethylene glycol, polypropylene glycol, propylene glycol), and mixtures thereof in suitable proportions. Exemplary solvates include, but are not limited to, hydrates, ethanolates, methanolates, isopropanolates and mixtures thereof. Methods of solvation are generally known to those of ordinary skill in the art.

As used herein, “pharmaceutically acceptable salt” refers to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like. The pharmaceutically acceptable salts of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 18th Edition, Mack Publishing Company, Easton, Pa. (1990), the disclosure of which is hereby incorporated by reference.

As used herein, the term “alkyl” unless otherwise specified refers to both branched and straight chain aliphatic (non-aromatic) hydrocarbons which may be primary, secondary, and/or tertiary hydrocarbons typically having 1 to 32 carbon atoms (e.g., C₁, C₂, C₃, C₄, C₅, C₆, C₇, C₈, C₉, C₁₀, C₁₁, C₁₂, C₁₃, C₁₄, etc.) and specifically includes, but is not limited to, saturated alkyl groups such as methyl, ethyl, propyl, isopropyl, butyl, isobutyl, t-butyl, pentyl, isopentyl, neopentyl, hexyl, isohexyl, 3-methylpentyl, 2,2-dimethylbutyl, 2,3-dimethylbutyl, 2-ethylhexyl, heptyl, octyl, nonyl, 3,7-dimethyloctyl, decyl, undecyl, dodecyl, tridecyl, tetradecyl, pentadecyl, hexadecyl, heptadecyl, octadecyl, nonadecyl, eicosyl, guerbet-type alkyl groups (e.g., 2-methylpentyl, 2-ethylhexyl, 2-proylheptyl, 2-butyloctyl, 2-pentylnonyl, 2-hexyldecyl, 2-heptylundecyl, 2-octyldodecyl, 2-nonyltridecyl, 2-decyltetradecyl, and 2-undecylpentadecyl), as well as unsaturated alkenyl and alkynyl variants such as vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, oleyl, linoleyl, and the like.

The term “cycloalkyl” refers to cyclized alkyl groups. Exemplary cycloalkyl groups include, but are not limited to, saturated cycloalkyl groups such as cyclopropyl, cyclobutyl, cyclopentyl, cyclohexyl, norbornyl, and adamantyl, branched cycloalkyl groups such as 1-methylcyclopropyl and 2-methylcyclopropyl, and cycloalkenyl groups such as cyclobutenyl, cyclopentenyl, and cyclohexenyl.

The term “aryl” means a carbocyclic aromatic monocyclic group containing 6 carbon atoms which may be further fused to a second 5- or 6-membered carbocyclic group which may be aromatic, saturated or unsaturated. Aryl includes, but is not limited to, phenyl, anthracenyl, indanyl, 1-naphthyl, 2-naphthyl, and tetrahydronaphthyl. The fused aryls may be connected to another group either at a suitable position on the cycloalkyl/cycloalkenyl ring or the aromatic ring.

The term “arylalkyl”, as used herein, refers to a straight or branched chain alkyl moiety (as defined above) that is substituted by an aryl group (as defined above), examples of which include, but are not limited to, benzyl, phenethyl, 2-methylbenzyl, 3-methylbenzyl, 4-methylbenzyl, 2,4-dimethylbenzyl, 2-(4-ethylphenyl)ethyl, 3-(3-propylphenyl) propyl, and the like.

The term “alkoxy” refers to an —O-alkyl group. Example alkoxy groups include, but are not limited to, methoxy, ethoxy, propoxy (e.g., n-propoxy and isopropoxy), and t-butoxy.

As used herein, the term “heterocycle” or “heterocyclyl” is intended to mean a stable 3-, 4-, 5-, 6-, or 7-membered monocyclic, 7-, 8-, 9-, 10-, or 11-membered bicyclic, or 7-, 8-, 9-, 10-, 11-, 12-, 13-, or 14-membered polycyclic heterocyclic ring that is saturated, partially unsaturated, or fully unsaturated, and that contains carbon atoms and 1, 2, 3 or 4 heteroatoms independently selected from the group consisting of N, O and S; and including any bicyclic or polycyclic group in which any of the above-defined heterocyclic rings is fused to a carbocyclic ring, the carbocyclic ring being either saturated, unsaturated, or aromatic (e.g., a benzene ring). The nitrogen and sulfur heteroatoms may optionally be oxidized (i.e., N→0 and S(O)ₚ, wherein p is 0, 1 or 2). The nitrogen atom may be substituted or unsubstituted (i.e., N or NR wherein R is H or another substituent, if defined). The heterocyclic ring may be attached to its pendant group at any heteroatom or carbon atom that results in a stable structure. The heterocyclic rings described herein may be substituted on carbon or on a nitrogen atom if the resulting compound is stable. A nitrogen in the heterocycle may optionally be quaternized. It is preferred that when the total number of S and O atoms in the heterocycle exceeds 1, then these heteroatoms are not adjacent to one another. It is preferred that the total number of S and O atoms in the heterocycle is not more than 1. When the term “heterocycle” is used, it is intended to include “heteroaryl” (which will be defined below).

Examples of heterocycles include, but are not limited to, acridinyl, azetidinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furyl, furazanyl, imidazolidinyl, imidazolinyl, imidazolyl, 1H-indazolyl, imidazolopyridinyl, indolenyl, indolinyl, indolizinyl, indolyl (e.g., 1H-indolyl), isatinoyl, isobenzofuranyl, isochromanyl, isoindazolyl, isoindolinyl, isoindolyl, isoquinolinyl, isothiazolyl, isothiazolopyridinyl, isoxazolyl, isoxazolopyridinyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolopyridinyl, oxazolidinylperimidinyl, oxindolyl, pyrimidinyl, phenanthridinyl, phenanthrolinyl, phenazinyl, phenothiazinyl, phenoxathiinyl, phenoxazinyl, phthalazinyl, piperazinyl, homopiperazinyl, piperidinyl, piperidonyl, 4-piperidonyl, piperonyl, pteridinyl, purinyl, pyranyl, pyrazinyl, pyrazolidinyl, pyrazolinyl, pyrazolopyridinyl, pyrazolyl, pyridazinyl, pyridooxazolyl, pyridoimidazolyl, pyridothiazolyl, pyridinyl, pyrimidinyl, pyrrolidinyl, pyrrolinyl, 2-pyrrolidonyl, 2H-pyrrolyl, pyrrolyl, quinazolinyl, quinolinyl, 4H-quinolizinyl, quinoxalinyl, quinuclidinyl, tetrazolyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thiazolopyridinyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. Also included are fused ring and spiro compounds containing, for example, the above heterocycles. Examples of 5- to 10-membered heterocycles include, but are not limited to, pyridinyl, thienyl, pyrrolyl, furyl, pyrazolyl, pyrazinyl, piperazinyl, piperidinyl, imidazolyl, imidazolidinyl, indolyl, tetrazolyl, isoxazolyl, morpholinyl, oxazolyl, oxadiazolyl, oxazolidinyl, tetrahydrofuranyl, thiadiazinyl, thiadiazolyl, thiazolyl, triazinyl, triazolyl, benzimidazolyl, IH-indazolyl, benzofuranyl, benzothiofuranyl, benztetrazolyl, benzo triazolyl, benzisoxazolyl, benzoxazolyl, oxindolyl, benzoxazolinyl, benzthiazolyl, benzisothiazolyl, isatinoyl, isoquinolinyl, octahydroisoquinolinyl, tetrahydroisoquinolinyl, tetrahydroquinolinyl, isoxazolopyridinyl, quinazolinyl, quinolinyl, isothiazolopyridinyl, thiazolopyridinyl, oxazolopyridinyl, imidazolopyridinyl, and pyrazolopyridinyl. Examples of 5- to 6-membered heterocycles include, but are not limited to, pyridinyl, furyl, thienyl, pyrrolyl, pyrazolyl, pyrazinyl, piperazinyl, piperidinyl, imidazolyl, imidazolidinyl, indolyl, tetrazolyl, isoxazolyl, mor-pholinyl, oxazolyl, oxadiazolyl, oxazolidinyl, tetrahydrofuranyl, thiadiazinyl, thiadiazolyl, thiazolyl, triazinyl, and triazolyl. Also included are fused ring and spiro compounds containing, for example, the above heterocycles. Examples of a bicyclic heterocyclic group include, but are not limited to, quinolinyl, isoquinolinyl, phthalazinyl, quinazolinyl, indolyl, isoindolyl, indolinyl, 1H-indazolyl, benzimidazolyl, 1,2,3,4-tetrahydroquinolinyl, 1,2,3,4-tetrahydroisoquinolinyl, 5,6,7,8-tetrahydro-quinolinyl, 2,3-dihydrobenzofuranyl, chromanyl, 1,2,3,4-tetrahydroquinoxalinyl, and 1,2,3,4-tetrahydro-quinazolinyl.

The term “heteroaryl” is intended to mean stable monocyclic and polycyclic aromatic hydrocarbons that include at least one heteroatom ring member such as sulfur, oxygen, or nitrogen. Heteroaryl groups are heterocyclyl groups which are aromatic, and may include, without limitation, pyridyl, pyrrolyl, pyrimidinyl, pyrazinyl, pyridazinyl, triazinyl, furyl, quinolyl, isoquinolyl, thienyl, imidazolyl, thiazolyl, indolyl (e.g., 1H-indolyl), pyrroyl, oxazolyl, benzofuryl, benzothienyl, benzthiazolyl, isoxazolyl, pyrazolyl, triazolyl, tetrazolyl, indazolyl (e.g., 1H-indazolyl), 1,2,4-thiadiazolyl, isothiazolyl, purinyl, carbazolyl, benzimidazolyl, indolinyl, benzodioxolanyl, and benzodioxane. Heteroaryl groups may be substituted or unsubstituted. The nitrogen atom may be substituted or unsubstituted (i.e., N or NR wherein R is H or another substituent, if defined). The nitrogen and sulfur heteroatoms may optionally be oxidized (i.e., N→0 and S(O)ₚ, wherein p is 0, 1 or 2).

The term “halo” or “halogen” includes fluoro, chloro, bromo and iodo.

As used herein, the term “substituted” refers to at least one hydrogen atom that is replaced with a non-hydrogen group, provided that normal valencies are maintained and that the substitution results in a stable compound. When a substituent is noted as “optionally substituted”, the substituent(s) are selected from alkyl, halo (e.g., chloro, bromo, iodo, fluoro), hydroxyl, alkoxy, oxo, alkanoyl, aryloxy, alkanoyloxy, amino (—NH₂), alkylamino (—NHalkyl), cycloalkylamino (—NHcycloalkyl), arylamino (—NHaryl), arylalkylamino (—NHarylalkyl), disubstituted amino (e.g., in which the two amino substituents are selected from alkyl, aryl or arylalkyl, including substituted variants thereof, with specific mention being made to dimethylamino), alkanoylamino, aroylamino, arylalkanoylamino, thiol, alkylthio, arylthio, arylalkylthio, alkylthiono, arylthiono, arylalkylthiono, alkylsulfonyl, arylsulfonyl, arylalkylsulfonyl, sulfonamide (e.g., —SO₂NH₂), substituted sulfonamide (e.g., —SO₂NHalkyl, —SO₂NHaryl, —SO₂NHarylalkyl, or cases where there are two substituents on one nitrogen selected from alkyl, aryl, or alkylalkyl), nitro, cyano, carboxy, unsubstituted amide (i.e. —CONH₂), substituted amide (e.g., —CONHalkyl, —CONHaryl, —CONHarylalkyl or cases where there are two substituents on one nitrogen selected from alkyl, aryl, or alkylalkyl), alkoxycarbonyl, aryl, guanidine, heterocyclyl (e.g., pyridyl, furyl, morpholinyl, pyrrolidinyl, piperazinyl, indolyl, imidazolyl, thienyl, thiazolyl, pyrrolidyl, pyrimidyl, piperidinyl, homopiperazinyl), and mixtures thereof. The substituents may themselves be optionally substituted, and may be either unprotected, or protected as necessary, as known to those skilled in the art, for example, as taught in Greene, et al., “Protective Groups in Organic Synthesis”, John Wiley and Sons, Second Edition, 1991, hereby incorporated by reference in its entirety.

In cases wherein there are nitrogen atoms (e.g., amines) on compounds of the present disclosure, these may be converted to N-oxides by treatment with an oxidizing agent (e.g., mCPBA and/or hydrogen peroxides) to afford other compounds of this disclosure. Thus, shown and claimed nitrogen atoms are considered to cover both the shown nitrogen and its N-oxide (NO) derivative.

### Compounds

In a first aspect, the present disclosure provides a compound of formula (I),

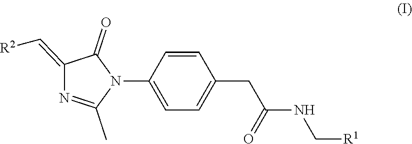

a salt thereof, a solvate thereof, a tautomer thereof, a stereoisomer thereof, or a mixture thereof;
wherein:
R¹ is selected from the group consisting of an optionally substituted alkyl, an optionally substituted cycloalkyl, an optionally substituted arylalkyl, and an optionally substituted aryl, and
R² is an optionally substituted heterocycle.

In some embodiments, R¹ is an aromatic moiety (e.g., aryl or heteroaryl) and a spacing between the aromatic moiety and the amide to which it is attached is 1-5 carbons, preferably 2, 3, or 4 carbons. In a preferred embodiment, a spacing between the aromatic moiety and the amide to which it is attached is 1 carbon, as depicted in formula (I). R¹ may be an optionally substituted monocyclic aryl or heteroaryl group or an optionally substituted bicyclic aryl or heteroaryl group, preferably R¹ is an optionally substituted monocyclic aryl. In some embodiments, R¹ may contain anywhere from 4 to 10 total carbon atoms, including any carbon atoms of substituents present, preferably 4 to 8 total carbon atoms, preferably 4 to 7 carbon atoms.

When R¹ is an optionally substituted aryl group, it is preferred that R¹ is phenyl (unsubstituted) or a substituted phenyl group. When R¹ is a substituted phenyl group, the phenyl moiety is preferably substituted with one or more substituents selected from a halo, preferably chloro or fluoro, preferably a chloro; an alkoxy, preferably a C₁ to C₄ alkoxy, preferably a C₁ to C₃ alkoxy, preferably a C₁ to C₂ alkoxy (e.g., methoxy or ethoxy), preferably a C₁ alkoxy (e.g., methoxy); and an alkyl, preferably a C₁ to C₄ alkyl, preferably a C₁ to C₃ alkyl, preferably a C₁ to C₂ alkyl (e.g., methyl or ethyl), preferably a C₁ alkyl (e.g., methyl). In some embodiments, when R¹ is a substituted phenyl, the phenyl group is substituted (at least) in the 4-position (para to the connection to the rest of the compound of formula (I)), preferably the phenyl group contains a single substituent at the 4-position, and R¹ is a 4-halophenyl, a 4-alkoxyphenyl, or 4-alkylphenyl, preferably 4-chlorophenyl, 4-methoxyphenyl, or 4-methylphenyl. In a most preferred embodiment, R¹ is a phenyl, as depicted in formula (Ia).

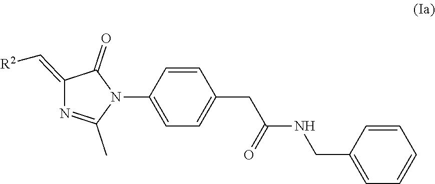

In some embodiments, R² is an aromatic moiety (e.g., aryl or heteroaryl) with a direct bond between the aromatic moiety and the double bond to which it is attached, such that the double bond is in conjugation with the aromatic group of R². In some embodiments, R² may be an optionally substituted monocyclic aryl or heteroaryl group or an optionally substituted bicyclic aryl or heteroaryl group, preferably R² is an optionally substituted monocyclic heteroaryl group. In some embodiments, R² may contain anywhere from 4 to 10 total carbon atoms, including any carbon atoms of substituents present, preferably 5 to 9 total carbon atoms, preferably 7 to 8 carbon atoms.

When R² is an optionally substituted heteroaryl group, it is preferred that R² contains a 5-membered ring with a nitrogen, oxygen, or sulfur containing heteroaryl substituent. Examples of 5-membered ring heteroaryl groups include but are not limited to furyl, imidazolyl, thiazolyl, thienyl, oxazolyl, isoxazolyl, triazolyl, pyrazolyl, isothiazolyl, or pyrroyl. In a preferred embodiment, the 5-membered ring heteroaryl groups are optionally substituted with halo, preferably chloro or fluoro, preferably a chloro; an alkoxy, preferably a C₁ to C₄ alkoxy, preferably a C₁ to C₃ alkoxy, preferably a C₁ to C₂ alkoxy (e.g., methoxy or ethoxy), preferably a C₁ alkoxy (e.g., methoxy); and an alkyl, preferably a C₁ to C₄ alkyl, preferably a C₁ to C₃ alkyl, preferably a C₁ to C₂ alkyl (e.g., methyl or ethyl), preferably a C₁ alkyl (e.g., methyl). In some embodiments, R² is an alkyl substituted furyl, imidazolyl, thiazolyl, thienyl, oxazolyl, isoxazolyl, triazolyl, pyrazolyl, isothiazolyl, or pyrroyl. In a most preferred embodiment, R² is a methyl substituted furyl as depicted in formula (Ib). In formula (Ib) the methyl substitution is depicted on the 2 carbon in the furyl ring, however, the substitution could also or instead take place at the carbons 3 or 4.

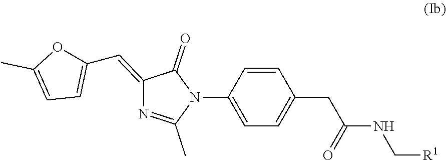

When R² is an optionally substituted heteroaryl group, it is preferred that R² contains a 6-membered ring with a nitrogen, oxygen, or sulfur containing heteroaryl substituent. Examples of 6-membered ring heteroaryl groups include but are not limited to pyridyl, pyrimidinyl, pyrazinyl, pyridazinyl, or triazinyl. In a preferred embodiment, the 6-membered ring heteroaryl groups are optionally substituted with halo, preferably chloro or fluoro, preferably a chloro; an alkoxy, preferably a C₁ to C₄ alkoxy, preferably a C₁ to C₃ alkoxy, preferably a C₁ to C₂ alkoxy (e.g., methoxy or ethoxy), preferably a C₁ alkoxy (e.g., methoxy); and an alkyl, preferably a C₁ to C₄ alkyl, preferably a C₁ to C₃ alkyl, preferably a C₁ to C₂ alkyl (e.g., methyl or ethyl), preferably a C₁ alkyl (e.g., methyl). In some embodiments, R² is an alkyl substituted pyridyl, pyrimidinyl, pyrazinyl, pyridazinyl, or triazinyl.

The compound of formula (I) may be selected from the group consisting of formulas (Ic)-(Iz). In a most preferred embodiment, the compound is of formula (Id).

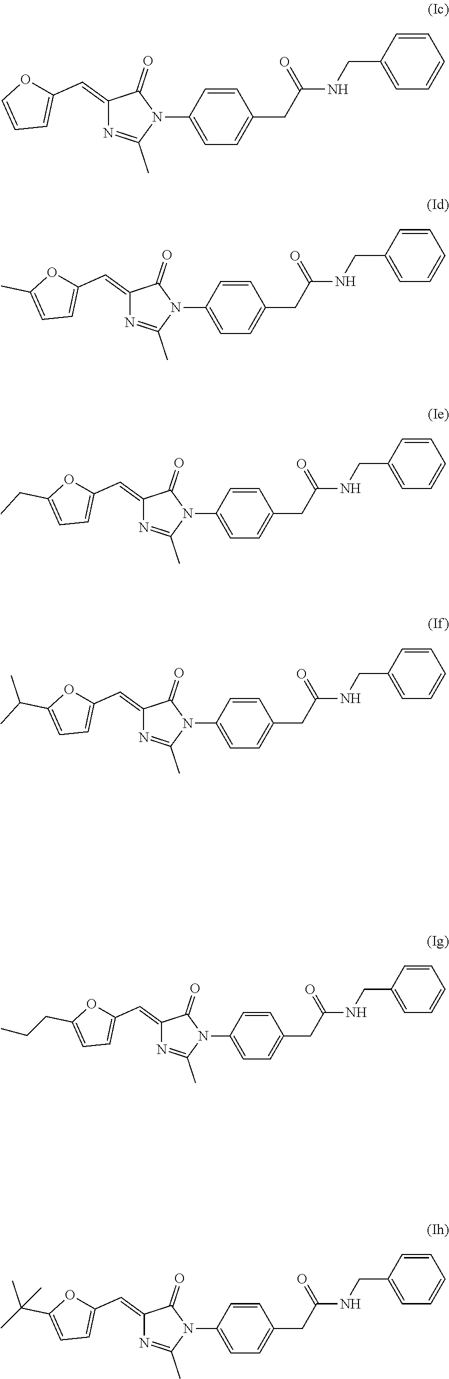 [SMILES: CC1=N/C(=C\c2ccco2)C(=O)N1c1ccc(CC(=O)NCc2ccccc2)cc1 ; CC1=N/C(=C\c2ccc(C)o2)C(=O)N1c1ccc(CC(=O)NCc2ccccc2)cc1 ; CCc1ccc(/C=C2\N=C(C)N(c3ccc(CC(=O)NCc4ccccc4)cc3)C2=O)o1 ; CC1=N/C(=C\c2ccc(C(C)C)o2)C(=O)N1c1ccc(CC(=O)NCc2ccccc2)cc1 ; CCCc1ccc(/C=C2\N=C(C)N(c3ccc(CC(=O)NCc4ccccc4)cc3)C2=O)o1 ; CC1=N/C(=C\c2ccc(C(C)(C)C)o2)C(=O)N1c1ccc(CC(=O)NCc2ccccc2)cc1]

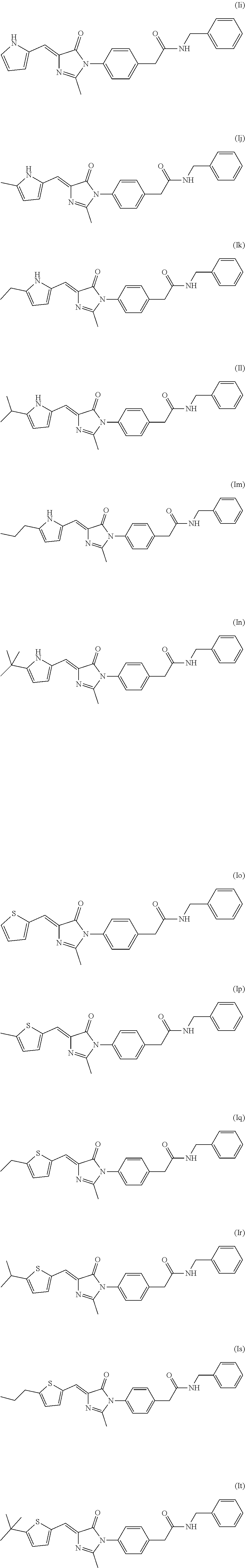

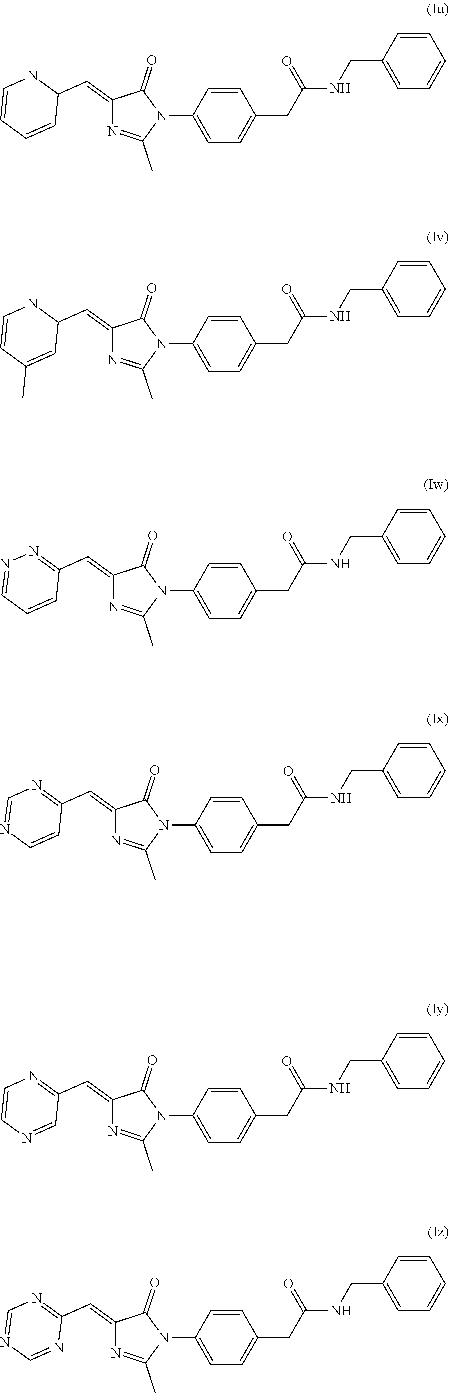

The compounds of the present disclosure may be prepared by methods known to those of ordinary skills in the art. The following methods set forth below are provided for illustrative purposes and are not intended to limit the scope of the disclosure.

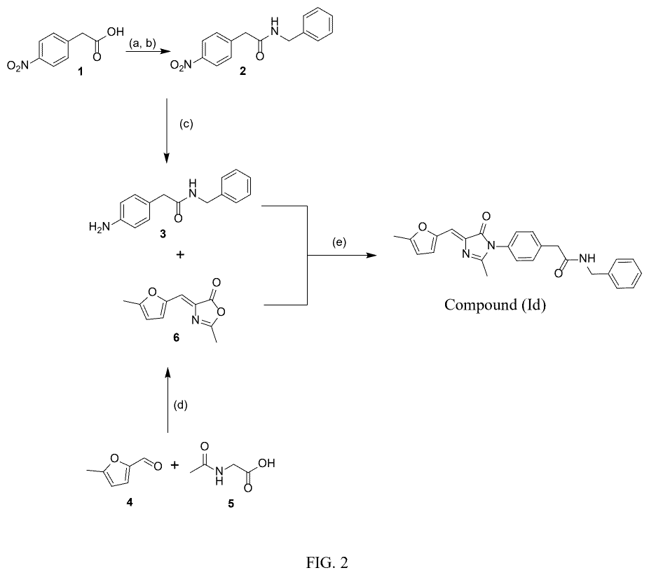

The compounds of formula (I) may, for example, be synthesized by a cyclocondensation reaction of amine (3) and oxazolone (6) starting materials, an embodiment of which is shown in the synthetic route illustrated in FIG. 2. FIG. 2 depicts a method of making a compound of formula (Id), however one of ordinary skill in the art would recognize that the materials could be modified to produce a compound with a different structure.

The amine (3) starting material can be made by converting a carboxylic acid (1) to a corresponding acyl chloride, as depicted in step (a). The acyl chloride can be made by any method known in the art, including but not limited to reacting the carboxylic acid (1) with a chloride salt, such as cobalt (II) chloride, in a polar aprotic solvent such as dimethylformamide. The resulting acyl chloride can then be directly reacted with an amine compound in the presence of a base, such as N,N-Diisopropylethylamine, to form an intermediate (2), as shown in step (b). In FIG. 2, the amine compound is benzylamine to provide the intermediate (2), however one of ordinary skill in the art would recognize that the amine compound could be modified in order to have a different R¹ substituent. In FIG. 2, R¹ is a phenyl. The intermediate (2) can further be reduced to form the amine (3) starting material as shown in step (c) by any method known in the art, including but not limited to hydrogenation over palladium on carbon.

The oxazolone (6) starting material can be made by reacting an aldehyde (4) and a carboxylic acid, as shown in step (d). The reaction can be performed by any method known in the art including but not limited to reacting the aldehyde in sodium acetate in anhydrous acetic acid. One of ordinary skill in the art would recognize that the aldehyde (4) could be modified in order to have a different R² substituent. In FIG. 2, R² is a methyl substituted furan. The amine (3) and oxazolone (6) starting materials can then be reacted by heating in an amine solvent such as pyridine to form the compound (Id).

The progress of any such reactions may be monitored by methods known to those of ordinary skill in the art, such as thin layer chromatography, gas chromatography, nuclear magnetic resonance, infrared spectroscopy, and high pressure liquid chromatography combined with ultraviolet detection or mass spectroscopy. The compounds of formula (I) may be isolated and purified by methods known to those of ordinary skill in the art, such as one or more of crystallization, precipitation, filtration, solvent evaporation, aqueous work-up, solvent extraction, drying, distillation, column chromatography, high pressure liquid chromatography (HPLC), lyophilization, and the like.

Of course, it should be understood that the compounds of formula (I) may be synthesized through various other synthetic schemes, reaction types and conditions, and isolation/purification procedures and still be considered a part of the present disclosure.

In a preferred embodiment, the compound of formula (I) has a maximum kinetic aqueous solubility of greater than 50 μM, preferably 55 μM, 60 μM, 65 μM, 70 μM, or 75 μM, and up to 100 μM, preferably 90 μM, or 80 μM. The maximum kinetic aqueous solubility a method used in drug discovery to measure the rate at which a compound dissolves in a solvent, aqueous buffer system or bio-relevant media. Kinetic solubility assays typically involve adding a small amount of the compound of interest to a buffered solution or media and monitoring the concentration of the compound over time.

### Pharmaceutical Compositions

According to a second aspect, the present disclosure relates to a pharmaceutical composition which comprises a therapeutically effective amount of one or more of the compounds of formula (I), formulated together with one or more pharmaceutically acceptable carriers and/or excipients, and optionally, one or more additional therapeutic agents. As described in detail below, the pharmaceutical compositions of the present disclosure may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained release formulation; (3) topical application, for example, as a cream, ointment, or a controlled release patch or spray applied to the skin; (4) intravaginally or intrarectally, for example, as a pessary, cream or foam; (5) sublingually; (6) ocularly; (7) transdermally; or (8) nasally.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, a “composition” or a “pharmaceutical composition” refers to a mixture of an active ingredient(s) with other chemical components, such as pharmaceutically acceptable carriers and/or excipients. One purpose of a composition is to facilitate administration of the compounds disclosed herein in any of its embodiments to a subject. Pharmaceutical compositions of the present disclosure may be manufactured by processes well known in the art, e.g., by means of conventional mixing, dissolving, granulating, dragee-making, levigating, emulsifying, encapsulating, entrapping or lyophilizing processes. Depending on the intended mode of administration (e.g., oral, parenteral, or topical), the composition can be in the form of solid, semi-solid or liquid dosage forms, such as tablets, suppositories, pills, capsules, powders, liquids, or suspensions, preferably in unit dosage form suitable for single administration of a precise dosage.

The term “active ingredient” or “active compound”, as used herein, refers to an ingredient in the composition that is biologically active, for example, one or more compounds represented by formula (I), a salt thereof, a solvate thereof, a tautomer thereof, a stereoisomer thereof, or any mixtures thereof. In some embodiments, additional therapeutic agents, in addition to the compound of the current disclosure, may be incorporated into a pharmaceutical composition, for example, a second active ingredient which is chemically distinct from the compounds of formula (I).

When the compounds of formula (I) are administered as pharmaceuticals, to humans and animals, they can be given per se or as a pharmaceutical composition containing, for example, 0.1 to 99 wt. % of active ingredient(s) in combination with a pharmaceutically acceptable carrier and/or excipient. In some embodiments, the pharmaceutical composition comprises 0.01 to 99 wt. % of the compound of formula (I) relative to a total weight of the pharmaceutical composition. For example, the pharmaceutical composition may contain at least 0.01 wt. %, at least 0.05 wt. %, at least 0.1 wt. %, at least 0.5 wt. %, at least 5 wt. %, at least 10 wt. %, at least 15 wt. %, at least 20 wt. %, at least 25 wt. %, at least 30 wt. %, at least 35 wt. %, at least 40 wt. %, at least 45 wt. %, at least 50 wt. %, at least 55 wt. %, at least 60 wt. %, at least 65 wt. %, at least 70 wt. %, and up to 99 wt. %, up to 98 wt. %, up to 95 wt. %, up to 90 wt. %, up to 85 wt. %, up to 80 wt. %, up to 75 wt. % of the compound of formula (I) relative to a total weight of the pharmaceutical composition. In preferred embodiments, the pharmaceutical composition includes 5 to 15 wt. %, preferably 6 to 14 wt. %, preferably 7 to 13 wt. %, preferably 8 to 12 wt. %, preferably 9 to 11 wt. %, preferably 10 wt. % of the compound of formula (I), based on a total weight of the pharmaceutical composition. In a most preferred embodiment, the compound of formula (Id) is included in the pharmaceutical composition.

In some embodiments, the active ingredient of the current disclosure, e.g., the compound of formula (I), a salt thereof, a solvate thereof, a tautomer thereof, a stereoisomer thereof, or any mixtures thereof, may provide utility as an anticancer agent in reducing the viability of cancer cells derived from human cancer cell lines including, but not limited to, breast cancer cell lines (e.g., MDA-MB-231, MCF-7, SK-BR-3, T47D, VP303); stomach cancer cell lines (e.g., N87, SNU-16, SNU-5, SNU-1, KATO III, AGS); colon/colorectal cancer cell lines (e.g., HCT-116, CACO-2, HT-29, HCT15, MDST8, GP5d, DLD1, SW620, SW403, T84); leukemia cell lines (e.g., HL-60, CESS, CCRF-CEM, CEM/C1, KASUMI-1, ARH-77, MV4-11, K562, NB4, and KG1a cells,); liver cancer cell lines (e.g., HepG2, PLC/PRF/5, THLE-3, C3A, SNU-182, SNU-398, SNU-387, SNU-423, SNU-475, SNU-449, and Hep 3B2.1-7); lung cancer cell lines (e.g., A549, NCI-H460, SHP-77, COR-L23/R, NCI-H69/LX20); brain tumor cell lines (e.g., U251); ovarian cancer cell lines (e.g., NCI-ADR/RES, OVCAR-03, A2780, A2780cis, OV7, PEO23); prostate cancer cell lines (e.g., PC-3); renal cancer cell lines (e.g., 786-0); and skin cancer or melanoma cell lines (e.g., UACC-62, C32TG, A375, MCC26).

Preferably, the active ingredient of the current disclosure, e.g., the compound of formula (I), a salt thereof, a solvate thereof, a tautomer thereof, a stereoisomer thereof, or any mixtures thereof, provides utility as an anticancer agent in reducing the viability of cancer cells derived from human colon/colorectal cancer cell lines (e.g., HCT-116, CACO-2, HT-29) and leukemia cell lines (e.g., HL-60, CESS, CCRF-CEM, CEM/C1, KASUMI-1, ARH-77, MV4-11, K562, NB4, and KG1a cells,).

In some embodiments, a method for decreasing an amount of colorectal cancer cells includes contacting the compound of formula (I) with the colorectal cancer cells. In some embodiments, an amount of the compound of formula (I) contacted with the colorectal cancer cells is 1 to 100 μg per mL of the cell culture, preferably 10-90 μg per mL, 20-80 μg per mL, 30-70 μg per mL, or 40-60 μg per mL. In some embodiments, an amount of the compound of formula (I) contacted with the colorectal cancer cells is 1 to 2 μg per mL of the cell culture, preferably 1.1-1.9 μg per mL, 1.2-1.8 μg per mL, 1.3-1.7 μg per mL, or 1.4-1.6 μg per mL.

In some embodiments, a method for decreasing an amount of leukemia cancer cells includes contacting the compound of formula (I) with the leukemia cancer cells. In some embodiments, an amount of the compound of formula (I) contacted with the leukemia cancer cells is 1 to 100 μg per mL of the cell culture, preferably 10-90 μg per mL, 20-80 μg per mL, 30-70 μg per mL, or 40-60 μg per mL. In some embodiments, an amount of the compound of formula (I) contacted with the leukemia cancer cells is 0.1 to 1 μg per mL of the cell culture, preferably 0.2-0.9 μg per mL, 0.3-0.8 μg per mL, 0.4-0.7 μg per mL, or 0.5-0.6 μg per mL.

In some embodiments, the cancer cells are collected from a human patient who is at risk of having, is suspected of having, has been diagnosed with, or is being monitored for recurrence of at least one type of cancer, preferably colon/colorectal cancer or leukemia.

In some embodiments, the ability of the active ingredient to reduce the viability of cancer cells may be determined by contacting the pharmaceutical composition with the cancer cells and then performing cell viability assays. Methods of such assays include, but are not limited to, sulforhodamine-B (SRB) assay, ATP test, Calcein AM assay, clonogenic assay, ethidium homodimer assay, Evans blue assay, 2′,7′-dichlorofluorescin diacetate (DCFDA) staining assay, fluorescein diacetate hydrolysis/propidium iodide staining assay, annexin V/fluorescein isothiocyanate (FITC)/propidium iodide staining assay, flow cytometry, Formazan-based assays (MTT, XTT), green fluorescent protein assay, lactate dehydrogenase (LDH) assay, methyl violet assay, propidium iodide assay, Resazurin assay, trypan blue assay, 4′,6′-diamidino-2-phenylindole (DAPI) assay, TUNEL assay, and primary (1°) colonosphere formation assay. In a preferred embodiment, a MTT assay is used. In another preferred embodiment, a Resazurin assay is used.

As is well understood in the art, the IC₅₀ value of a compound/mixture is a concentration of that compound/mixture which causes the death of 50% of the cellular population to which the compound/mixture is added. In some embodiments, the IC₅₀ of the compound of formula (I), the salt thereof, the solvate thereof, the tautomer thereof, the stereoisomer thereof, or the mixture thereof against colorectal cancer cells, for example HCT-116, DLD1, and HT-29 cell lines, is less than 20 μM, preferably less than 15 μM, preferably less than 10 μM, preferably less than 9 μM, preferably less than 8 μM, preferably less than 7 μM, preferably less than 6 μM, preferably less than 5 μM, preferably less than 4 μM, preferably less than 3 μM, preferably less than 2.5 μM, preferably less than 2 μM, preferably less than 1.5 μM, preferably less than 1 μM, for example, from 0.8 to 2 μM, preferably from 0.9 to 1.5 μM, preferably from 1.0 to 1.4 μM, preferably from 1.1 to 1.4 μM, preferably from 1.3-1.4 μM.

In some embodiments, the IC₅₀ of the compound of formula (I), the salt thereof, the solvate thereof, the tautomer thereof, the stereoisomer thereof, or the mixture thereof against leukemia cells, preferably Myeloid leukemia cell lines, for example, HL60, MV4-11, K562, Kasumi-1, NB4, and KG1α cells, is less than 20 μM, preferably less than 15 μM, preferably less than 10 μM, preferably less than 9 UM, preferably less than 8 μM, preferably less than 7 μM, preferably less than 6 μM, preferably less than 5 μM, preferably less than 4 μM, preferably less than 3 μM, preferably less than 2.5 μM, preferably less than 2 μM, preferably less than 1.5 μM, preferably less than 1 μM, for example, from 0.01 to 2 μM, preferably from 0.05 to 1.5 μM, preferably from 0.1 to 1.0 μM, preferably from 0.2 to 0.9 μM, preferably from 0.3-0.8 μM, preferably from 0.4-0.7 μM, preferably from 0.5-0.6 μM.

In some embodiments, additional therapeutic agents in addition to the compound of the current disclosure may be incorporated into the pharmaceutical composition. In some embodiments, the pharmaceutical composition includes an additional therapeutic agent that is chemically distinct from the compound of formula (I), such as a chemotherapeutic agent or an anticancer agent, for the treatment or prevention of neoplasm, of tumor or cancer cell division, growth, proliferation and/or metastasis in the subject; induction of death or apoptosis of tumor and/or cancer cells; and/or any other forms of proliferative disorder.

The additional therapeutic agent may be an anticancer agent and may include, but is not limited to, at least one of a mitotic inhibitor; an alkylating agent; an antimetabolite; a cell cycle inhibitor; an enzyme; a topoisomerase inhibitor; a biological response modifier; an anti-hormone; a tubulin inhibitor; a tyrosine-kinase inhibitor; an antiangiogenic agent such as MMP-2, MMP-9 and COX-2 inhibitor; an anti-androgen; a platinum coordination complex (oxaliplatin, carboplatin); a substituted urea such as hydroxyurea; a methylhydrazine derivative; an adrenocortical suppressant, e.g., mitotane, aminoglutethimide; a hormone and/or hormone antagonist such as the adrenocorticosteriods (e.g., prednisone), progestins (e.g., hydroxyprogesterone caproate), an estrogen (e.g., diethylstilbestrol); an antiestrogen such as tamoxifen; androgen, e.g., testosterone propionate; and an aromatase inhibitor, such as anastrozole, and AROMASIN (exemestane).

Exemplary additional therapeutic agents include, but are not limited to, tubulin binding agents including paclitaxel, epothilone, docetaxel, discodermolide, etoposide, vinblastine, vincristine, teniposide, vinorelbine, and vindesine; tyrosine-kinase inhibitors including imatinib, nilotinib, dasatinib, bosutinib, ponatinib, and bafetinib; alkylating antineoplastic agents including busulfan, carmustine, chlorambucil, cyclophosphamide, cyclophosphamide, dacarbazine, ifosfamide, lomustine, mechlorethamine, melphalan, mercaptopurine, procarbazine; antimetabolites including cladribine, cytarabine, fludarabine, gemcitabine, pentostatin, 5-fluorouracil, clofarabine, capecitabine, methotrexate, thioguanine; cytotoxic antibiotics including daunorubicin, doxorubicin, idarubicin, mitomycin, actinomycin, epirubicin; topoisomerase inhibitors including irinotecan, mitoxantrone, topotecan, and mixtures thereof.

As used herein, the phrase “pharmaceutically acceptable carrier and/or excipient” means a pharmaceutically acceptable material, composition or vehicle, such as a liquid or solid filler, carrier, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, com oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations, such as cyclodextrins, liposomes, and micelle forming agents, e.g., bile acids, just to name a few.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the pharmaceutical compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Methods of preparing these pharmaceutical compositions include the step of bringing into association a compound of formula (I) with the pharmaceutically acceptable carrier and/or excipient, and, optionally, one or more accessory ingredients. In general, the compositions are prepared by uniformly and intimately bringing into association a compound of the present disclosure with liquid carriers, or finely divided solid carriers, or both, and then, if necessary, shaping the product.

Pharmaceutical compositions of the present disclosure suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of formula (I) as an active ingredient. A compound of formula (I) of the present disclosure may also be administered as a bolus, electuary or paste.

In solid dosage forms of the present disclosure for oral administration (capsules, tablets, pills, dragees, powders, granules, troches and the like), the active ingredient is mixed with one or more pharmaceutically acceptable carriers and/or excipients, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants (e.g., fatty acid esters of sorbitan and polyalkolyated fatty acid esters of sorbitan such as TWEEN 80, available from Sigma-Aldrich); (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface active or dispersing agent. Molded tablets may be made by molding in a suitable machine a mixture of the powdered compound moistened with an inert liquid diluent. The tablets, and other solid dosage forms of the pharmaceutical compositions of the present disclosure may optionally be scored or prepared with coatings and shells, such as enteric coatings and other coatings well known in the pharmaceutical formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacterium retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above described excipients.

Liquid dosage forms for oral administration of the compounds of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, com, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compound, may contain suspending agents as, for example, ethoxylated isostearyl alcohols, polyoxyethylene sorbitol and sorbitan esters (including polyoxyethylene fatty acid esters of sorbitan, e.g., TWEEN 80), microcrystalline cellulose, aluminum metahydroxide, bentonite, agar-agar and tragacanth, and mixtures thereof. In preferred embodiments, the pharmaceutical composition is in the form of a suspension, comprising, consisting of, or consisting essentially of the compound of formula (I) and the pharmaceutically acceptable carrier and/or excipient, which is preferably a suspending agent (preferably a polyoxyethylene sorbitan ester, preferably a polyoxyethylene fatty acid ester of sorbitan, e.g., TWEEN 80) in an inert diluent (preferably water). Preferably the content of the suspending agent in the suspension ranges from 0.01 to 1 wt. %, preferably 0.05 to 0.8 wt. %, preferably 0.1 to 0.6 wt. %, preferably 0.5 wt. %, based on a total weight of the suspension.

Formulations of the pharmaceutical compositions of the disclosure for rectal or vaginal administration may be presented as a suppository, which may be prepared by mixing one or more compounds of formula (I) with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound(s).

Formulations of the pharmaceutical compositions which are suitable for vaginal administration also include pessaries, tampons, creams, gels, pastes, foams or spray formulations containing such carriers as are known in the art to be appropriate.

Dosage forms for the topical or transdermal administration of a compound of this disclosure include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this disclosure, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present disclosure to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this disclosure.

Pharmaceutical compositions of this disclosure suitable for parenteral administration comprise one or more compounds of formula (I) in combination with one or more pharmaceutically acceptable sterile isotonic aqueous or non-aqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants (e.g., TWEEN 80).

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the pharmaceutical compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsuled matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly (anhydrides). Depot injectable formulations are also prepared by entrapping the drug in liposomes or microemulsions which are compatible with body tissue.

In some embodiments, the pharmaceutical composition contains 1 to 99.9 wt. %, preferably 5 to 95 wt. %, preferably 10 to 90 wt. %, preferably 15 to 85 wt. %, preferably 20 to 80 wt. %, preferably 30 to 75 wt. %, preferably 40 to 70 wt. %, preferably 50 to 65 wt. % of the pharmaceutically acceptable carrier and/or excipient, relative to a total weight of the pharmaceutical composition.

### Therapeutic Applications and Methods

According to a third aspect, the present disclosure relates to a method for treating a proliferative disorder. The method involves administering a therapeutically effective amount of one or more compounds of formula (I) per se, or a pharmaceutical composition of the second aspect to a subject.

In some embodiments, the proliferative disorder is cancer. Types of cancers that may be treated with the compounds of this disclosure include, but are not limited to, brain cancers, skin cancers, bladder cancers, ovarian cancers, breast cancers, gastric cancers, pancreatic cancers, prostate cancers, colon/colorectal cancers, blood cancers, lung cancers and bone cancers. In some embodiments, the compounds of this disclosure can be used for the treatment of any cancer type that fails to undergo apoptosis in a patient. This includes but is not limited to solid tumors, including but not limited to carcinomas; sarcomas including Kaposi's sarcoma; erythroblastoma; glioblastoma; meningioma; astrocytoma; melanoma; and myoblastoma. Treatment or prevention of non-solid tumor cancers, such as leukemia, is also contemplated by this invention.

Examples of such cancer types include neuroblastoma, intestine carcinoma such as rectum carcinoma, colon carcinoma, familiar adenomatous polyposis carcinoma and hereditary non-polyposis colorectal cancer, esophageal carcinoma, labial carcinoma, larynx carcinoma, hypopharynx carcinoma, tong carcinoma, salivary gland carcinoma, gastric carcinoma, adenocarcinoma, medullary thyroid carcinoma, papillary thyroid carcinoma, renal carcinoma, kidney parenchymal carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors such as glioblastoma, astrocytoma, meningioma, medulloblastoma and peripheral neuroectodermal tumors, Hodgkin lymphoma, non-Hodgkin lymphoma, Bur-kitt lymphoma, acute lymphatic leukemia (ALL), chronic lymphatic leukemia (CLL), acute myeloid leukemia (AML), chronic myeloid leukemia (CML), adult T-cell leukemia lymphoma, diffuse laige B-cell lymphoma (DLBCL), hepatocellular carcinoma, gall bladder carcinoma, bronchial carcinoma, small cell lung carcinoma, non-small cell lung carcinoma, multiple myeloma, basalioma, teratoma, retinoblastoma, choroid melanoma, seminoma, rhabdomyosarcoma, craniopharyngioma, osteosarcoma, chondrosarcoma, myosarcoma, liposarcoma, fibrosarcoma, Ewing sarcoma and plasmocytoma. In preferred embodiments, the cancer is colon or colorectal cancer or leukemia, specifically myeloid leukemia.

As used herein, the terms “treat”, “treatment”, and “treating” in the context of the administration of a therapy to a subject in need thereof refers to the reduction or inhibition of the progression and/or duration of a disease (e.g., cancer), the reduction or amelioration of the severity of the disease, the amelioration of one or more symptoms thereof resulting from the administration of one or more therapies, preventing the disease from occurring in a subject that may be predisposed to the disease but does not yet experience or exhibit symptoms of the disease (prophylactic treatment), slowing or arresting disease development, ameliorating the disease, providing relief from the symptoms or side-effects of the disease (including palliative treatment), and causing regression of the disease. Specific to cancer, and in particular colon or colorectal cancer, these terms may refer to: (1) a stabilization, reduction (e.g., by more than 10%, 20%, 30%, 40%, 50%, preferably by more than 60% of the population of cancer cells and/or tumor size before administration), or elimination of the cancer cells, (2) inhibiting cancerous cell division and/or cancerous cell proliferation, (3) relieving to some extent (or, preferably, eliminating) one or more symptoms associated with a pathology related to or caused in part by unregulated or aberrant cellular division, (4) an increase in disease-free, relapse-free, progression-free, and/or overall survival, duration, or rate, (5) a decrease in hospitalization rate, (6) a decrease in hospitalization length, (7) eradication, removal, or control of primary, regional and/or metastatic cancer, (8) a stabilization or reduction (e.g., by at least 10%, 20%, 30%, 40%, 50%, 60%, 70%, preferably at least 80% relative to the initial growth rate) in the growth of a tumor or neoplasm, (9) an impairment in the formation of a tumor, (10) a reduction in mortality, (11) an increase in the response rate, the durability of response, or number of patients who respond or are in remission, (12) the size of the tumor is maintained and does not increase or increases by less than 10%, preferably less than 5%, preferably less than 4%, preferably less than 2%, (13) a decrease in the need for surgery (e.g., colectomy, mastectomy), and (14) preventing or reducing (e.g., by more than 10%, more than 30%, preferably by more than 60% of the population of metastasized cancer cells before administration) the metastasis of cancer cells.

The term “subject” and “patient” are used interchangeably. As used herein, they refer to any subject for whom or which therapy, including with the compositions according to the present disclosure is desired. In most embodiments, the subject is a mammal, including but not limited to a human, a non-human primate such as a chimpanzee, a domestic livestock such as a cattle, a horse, a swine, a pet animal such as a dog, a cat, and a rabbit, and a laboratory subject such as a rodent, e.g., a rat, a mouse, and a guinea pig. In preferred embodiments, the subject is a human.

The subject may be any subject already with the disease, a subject which does not yet experience or exhibit symptoms of the disease, or a subject predisposed to the disease. In preferred embodiments, the subject is a person who is predisposed to cancer, e.g., a person with a family history of cancer. Women who have (i) certain inherited genes (e.g., mutated BRCA1 and/or mutated BRCA2), (ii) been taking estrogen alone (without progesterone) after menopause for many years (at least 5, at least 7, or at least 10), and/or (iii) been taking fertility drug clomiphene citrate, are at a higher risk of contracting breast cancer. People who (i) consumes a diet high in salty and smoked foods and/or low in fruits and vegetables, (ii) had infection with Helicobacter pylori, and/or (iii) long-term stomach inflammation are at a higher risk of contracting stomach cancer. People who (i) had chemotherapy and radiation therapy for other cancers, (ii) has genetic disorders, such as Down syndrome, and/or (iii) exposure to certain chemicals, such as benzene are at a higher risk of contracting leukemia. People who (i) had inflammatory bowel disease, or a genetic syndrome such as familial adenomatous polyposis (FAP) and hereditary non-polyposis colorectal cancer (Lynch syndrome), and/or (ii) consumes a low-fiber and high-fat diet are at a higher risk of contracting colon cancer. Any subject with such predispositions, in combination with sound medical judgment, may be candidates for the treatment methods described herein.

In some embodiments, the subject has leukemia, stomach, colon, and/or breast cancer and is currently undergoing, or has completed one or more chemotherapy regimens. In some embodiments, the subject has been previously administered/treated with, or is being currently administered/treated with, a thymidylate synthase inhibitor (e.g., capecitabine, fluorouracil (5-FU)), a thymidine phosphorylase (TPase) inhibitor (e.g., tipiracil, trifluridine), topoisomerase I inhibitor (e.g., irinotecan), a DNA synthesis inhibitor (e.g., oxaliplatin), and/or a targeted therapy (e.g., cetuximab, bevacizumab, panitumumab, zivaflibercept, ramucirumab). In some embodiments, the subject has been previously administered/treated with, or is being currently administered/treated with, a tubulin binding drug such as paclitaxel, epothilone, docetaxel, discodermolide, etoposide, vinblastine, vincristine, teniposide, vinorelbine, and vindesine, and developed resistance to the tubulin binding drug. In some embodiments, the subject has been previously administered/treated with, or is being currently administered/treated with, a tyrosine-kinase inhibitor such as imatinib, nilotinib, dasatinib, bosutinib, ponatinib, and bafetinib, and developed drug resistance via (i) Bcr-Abl dependent mechanisms involving Bcr-Abl duplication, Bcr-Abl mutation, T315I mutation, and/or P-loop mutations, or (ii) Bcr-Abl Independent mechanisms involving drug efflux caused by P-glycoproteins, drug import by organic cation transporter 1, and/or alternative signaling pathway activation.

The terms “administer”, “administering”, “administration”, and the like, as used herein, refer to the methods that may be used to enable delivery of the active ingredient and/or the pharmaceutical composition to the desired site of biological action. Routes or modes of administration are as set forth herein. These methods include, but are not limited to, oral routes, intraduodenal routes, parenteral injection (including intravenous, subcutaneous, intraperitoneal, intramuscular, intravascular, or infusion), topical and rectal administration. Those of ordinary skill in the art are familiar with administration techniques that can be employed. In preferred embodiments, the active ingredient (e.g., the compound of formula (I)) or the pharmaceutical composition described herein are administered orally, preferably as an oral suspension.

The dosage amount and treatment duration are dependent on factors, such as bioavailability of a drug, administration mode, toxicity of a drug, gender, age, lifestyle, body weight, the use of other drugs and dietary supplements, the disease stage, tolerance and resistance of the body to the administered drug, etc., and then determined and adjusted accordingly. The terms “effective amount”, “therapeutically effective amount”, or “pharmaceutically effective amount” refer to that amount of the active ingredient being administered which will relieve to some extent one or more of the symptoms of the disease being treated. The result can be a reduction and/or alleviation of the signs, symptoms, or causes of a disease, or any other desired alteration of a biological system. An appropriate “effective amount” may differ from one individual to another. An appropriate “effective amount” in any individual case may be determined using techniques, such as a dose escalation study. Typically, an effective amount of the compound of formula (I) to treat cancers such as colon or colorectal cancer, in terms of mg of the compound of formula (I) per body weight of the subject (kg), ranges from 0.1 to 2,000 mg/kg, preferably 1 to 1,500 mg/kg, preferably 5 to 1,000 mg/kg, preferably 10 to 900 mg/kg, preferably 15 to 800 mg/kg, preferably 20 to 700 mg/kg, preferably 30 to 600 mg/kg, preferably 40 to 500 mg/kg, preferably 50 to 400 mg/kg, preferably 60 to 300 mg/kg, preferably 70 to 200 mg/kg, preferably 80 to 150 mg/kg, preferably 90 to 100 mg/kg.

Compounds of the disclosure may be useful for sensitizing cells to apoptotic signals. Thus, in some embodiments, the compounds of the disclosure are co-administered with radiation therapy or a second therapeutic agent with cytostatic or antineoplastic activity. Suitable cytostatic chemotherapy compounds include, but are not limited to (i) antimetabolites; (ii) DNA-fragmenting agents, (iii) DNA-crosslinking agents, (iv) intercalating agents (v) protein synthesis inhibitors, (vi) topoisomerase I poisons, such as camptothecin ortopotecan; (vii) topoisomerase II poisons, (viii) microtubule-directed agents, (ix) kinase inhibitors (x) miscellaneous investigational agents (xi) hormones, (xii) hormone antagonists, and (xii) targeted therapies. It is contemplated that compounds of the disclosure may be useful in combination with any known agents falling into the above 13 classes as well as any future agents that are currently in development. In particular, it is contemplated that compounds of the disclosure may be useful in combination with current Standards of Care as well as any that evolve over the foreseeable future. Specific dosages and dosing regimens would be based on physicians' evolving knowledge and the general skill in the art.

Examples of second therapeutic agents include, but are not limited to, a mitotic inhibitor; an alkylating agent; an antimetabolite; a cell cycle inhibitor; an enzyme; a topoisomerase inhibitor; a biological response modifier; an anti-hormone; a tubulin inhibitor; a tyrosine-kinase inhibitor; an antiangiogenic agent such as MMP-2, MMP-9 and COX-2 inhibitor; an anti-androgen; a platinum coordination complex (oxaliplatin, carboplatin); a substituted urea such as hydroxyurea; a methylhydrazine derivative; an adrenocortical suppressant, e.g., mitotane, aminoglutethimide; a hormone and/or hormone antagonist such as the adrenocorticosteriods (e.g., prednisone), progestins (e.g., hydroxyprogesterone caproate), an estrogen (e.g., diethylstilbestrol); an antiestrogen such as tamoxifen; androgen, e.g., testosterone propionate; and an aromatase inhibitor, such as anastrozole, and AROMASIN (exemestane); a thymidylate synthase inhibitor; a thymidine phosphorylase (TPase) inhibitor; a DNA synthesis inhibitor; and/or a targeted therapy. Exemplary second therapeutic agents include, but are not limited to, tubulin binding agents including paclitaxel, epothilone, docetaxel, discodermolide, etoposide, vinblastine, vincristine, teniposide, vinorelbine, and vindesine; tyrosine-kinase inhibitors including imatinib, nilotinib, dasatinib, bosutinib, ponatinib, and bafetinib; alkylating antineoplastic agents including busulfan, carmustine, chlorambucil, cyclophosphamide, cyclophosphamide, dacarbazine, ifosfamide, lomustine, mechlorethamine, melphalan, mercaptopurine, procarbazine; antimetabolites including cladribine, cytarabine, fludarabine, gemcitabine, pentostatin, 5-fluorouracil, clofarabine, capecitabine, methotrexate, thioguanine; cytotoxic antibiotics including daunorubicin, doxorubicin, idarubicin, mitomycin, actinomycin, epirubicin; topoisomerase inhibitors including irinotecan, mitoxantrone, topotecan; thymidine phosphorylase (TPase) inhibitors such as tipiracil and trifluridine; DNA synthesis inhibitors such as oxaliplatin; targeted therapies such as cetuximab, bevacizumab, panitumumab, zivaflibercept, ramucirumab; and mixtures thereof.

The combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single dosage form having a fixed ratio of each therapeutic agent or in multiple, single dosage forms for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. Combination therapy also can embrace the administration of the therapeutic agents as described above in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

A treatment method may comprise administering the compound of formula (I) or a pharmaceutical composition containing the compound of formula (I) of the current disclosure in any of its embodiments as a single dose or multiple individual divided doses. In some embodiments, the composition is administered at various dosages (e.g., a first dose with an effective amount of 200 mg/kg and a second dose with an effective amount of 50 mg/kg). In some embodiments, the interval of time between the administration of the pharmaceutical composition and the administration of one or more second therapies may be about 1 to 5 minutes, 1 to 30 minutes, 30 minutes to 60 minutes, 1 hour, 1 to 2 hours, 2 to 6 hours, 2 to 12 hours, 12 to 24 hours, 1 to 2 days, 2 days, 3 days, 4 days, 5 days, 6 days, 7 days, 1 week, 2 weeks, 3 weeks, 4 weeks, 5 weeks, 6 weeks, 7 weeks, 8 weeks, 9 weeks, 10 weeks, 15 weeks, 20 weeks, 26 weeks, 52 weeks, 11 to 15 weeks, 15 to 20 weeks, 20 to 30 weeks, 30 to 40 weeks, 40 to 50 weeks, 1 month, 2 months, 3 months, 4 months, 5 months, 6 months, 7 months, 8 months, 9 months, 10 months, 11 months, 12 months, 1 year, 2 years, or any period of time in between. Preferably, the composition is administered once daily for at least 2 days, at least 5 days, at least 6 days, or at least 7 days. In some embodiments, the pharmaceutical composition and optionally one or more second therapies are administered less than 1 day, less than 1 week, less than 2 weeks, less than 3 weeks, less than 4 weeks, less than 1 month, less than 2 months, less than 3 months, less than 6 months, less than 1 year, less than 2 years, or less than 5 years apart.

The methods for treating cancer and other proliferative disorders described herein inhibit, remove, eradicate, reduce, regress, diminish, arrest or stabilize a cancerous tumor, including at least one of the tumor growth, tumor cell viability, tumor cell division and proliferation, tumor metabolism, blood flow to the tumor and metastasis of the tumor. In some embodiments, the size of a tumor, whether by volume, weight or diameter, is reduced after the treatment by at least 5%, at least 10%, at least 15%, at least 20%, at least 25%, at least 30%, at least 40%, at least 50%, at least 60%, at least 70%, at least 75%, at least 80%, at least 85%, at least 90%, at least 95%, at least 99%, or 100%, relative to the tumor size before treatment. In some embodiments, the size of a tumor after treatment is not reduced but is maintained at the same size as before treatment. Methods of assessing tumor size include, but are not limited to, CT scan, MRI, DCE-MRI and PET scan.

The method may further comprise measuring a concentration of a biomarker and/or detecting a mutation in a biomarker before and/or after the pharmaceutical composition comprising the compound of formula (I) of the present disclosure is administered. Generic cancer biomarkers include circulating tumor DNA (ctDNA) and circulating tumor cells (CTC). Exemplary biomarkers for colon cancer include, without limitation, carcinoembryonic antigen (CEA), carbohydrate antigen 242 (CA 242), CA 195, CA 19-9, MSI, and 18qLOH. Exemplary biomarkers for breast cancer include, without limitation, BRCA1, BRCA2, HER-2, estrogen receptor, progesterone receptor, cancer antigen 15-3, cancer antigen 27.29, carcinoembryonic antigen, Ki67, cyclin D1, cyclin E, and ERB. Exemplary biomarkers for stomach cancer include, without limitation, carcinoembryonic antigen (CEA), CA19-9, carbohydrate antigen (CA) 72-4, alpha-fetoprotein, carbohydrate antigen (CA) 12-5, SLE, BCA-225, hCG, and pepsinogen I/II.

Potentially predictive cancer biomarkers include, without limitation, mutations in genes BRCA1 and BRCA2 for breast cancer, overexpressions of TYMS, mutations in genes p53 and KRAS for colon cancer, and high concentration levels of AFP, and overexpressions of HSP90a for liver cancer.

The mutation in the biomarker may be detected by procedures such as restriction fragment length polymorphism (RFLP), polymerase chain reaction (PCR) assay, multiplex ligation-dependent probe amplification (MLPA), denaturing gradient gel electrophoresis (DGGE), single-strand conformation polymorphism (SSCP), hetero-duplex analysis, protein truncation test (PTT), and oligonucleotide ligation assay (OLA). The procedures to detect the mutation are well-known to those of ordinary skill in the art.

The concentration level of the cancer biomarker in a sample (i.e., biological sample obtained from the subject in need of therapy including a single cell, multiple cells, fragments of cells, a tissue sample, and/or body fluid, for example red blood cells, white blood cells, platelets, hepatocytes, epithelial cells, endothelial cells, a skin biopsy, a mucosa biopsy, an aliquot of urine, saliva, whole blood, serum, plasma, lymph) may be measured for example by an immunoassay. Typical immunoassay methods include, without limitation, enzyme-linked immunosorbent assay (ELISA), enzyme-linked immunospot assay (ELISPOT), Western blotting, immunohistochemistry (IHC), immunocytochemistry, immunostaining, and multiple reaction monitoring (MRM) based mass spectrometric immunoassay. The protocol for measuring the concentration of the biomarker and/or detecting the mutation in the biomarker is known to those of ordinary skill, for example by performing the steps outlined in the commercially available assay kit sold by Sigma-Aldrich, Thermo Fisher Scientific, R & D Systems, ZeptoMetrix Inc., Cayman Inc., Abcam, Trevigen, Dojindo Molecular Technologies, Biovision, and Enzo Life Sciences.

In some embodiments, a concentration of the biomarker is measured before and after the administration. When the concentration of the biomarker is maintained, the method may further comprise increasing the effective amount of the compound of formula (I) by at least 5%, at least 10%, or at least 30%, and up to 80%, up to 60%, or up to 50% of an initial effective amount. The subject may be administered with the increased dosage for a longer period (e.g., 1 week more, 2 weeks more, or 2 months more) than the duration prescribed with the initial effective amount.

In some embodiments, the administration is stopped once the subject is treated.

The examples below are intended to further illustrate protocols for preparing, characterizing, and using the compounds of the present disclosure, and are not intended to limit the scope of the claims.

Where a numerical limit or range is stated herein, the endpoints are included. Also, all values and subranges within a numerical limit or range are specifically included as if explicitly written out.

The terms “comprise(s)”, “include(s)”, “having”, “has”, “can”, “contain(s)”, and variants thereof, as used herein, are intended to be open-ended transitional phrases, terms, or words that do not preclude the possibility of additional acts or structures. The present disclosure also contemplates other embodiments “comprising”, “consisting of” and “consisting essentially of”, the embodiments or elements presented herein, whether explicitly set forth or not.

As used herein, the words “a” and “an” and the like carry the meaning of “one or more.”

Numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

### EXAMPLES

### Example 1: Chemical Synthesis of Compound (Id)

The compound (Id) was synthesized following the method illustrated in FIG. 2. The synthesis of the desired 2-methyl[5-arylidene]1,4-dihydro-4H-imidazol-5-one derivative was achieved by cyclocondensation reaction of the amine 3 and oxazolone 6 starting materials. For the amine 3, the carboxylic acid 1 was activated by conversion to the corresponding acid chloride. Without isolation, the acid chlorides were reacted with benzylamine to provide the intermediate 2. Reduction of the nitro group to afford amine 3 was accomplished through hydrogenation.

The oxazolone 6 ((Z)-2-methyl-4-((5-methylfuran-2-yl)methylene) oxazol-5 (4H)-one) was achieved by cycloaddition reaction of the aldehyde 4 and N-acetylglycine 5.

A mixture of the aldehyde 4, N-acetylglycine 5 (1.3 eq) and freshly fused Na acetate (1 eq) in anhydrous acetic anhydride (minimum amount) was stirred at 130° C. for 13 h under inert atmosphere. After allowing the mixture to cool to room temperature, the mixture was cooled in ice bath and the precipitate was vacuum filtrated and washed with ice-cold water. The product was used as is for next step.

The compound (Id) (2-Methyl-4-(5-methyl-2-furyl)methylene-1-[4-(benzylaminocarbonylmethyl)phenyl]-5-oxo-1H-imidazole) was synthesized by the following. To a solution of the selected oxazolone 6 (10 mmol) in dry pyridine (10 mL) was added 2-(4-aminophenyl)-N-benzylacetamide (2.41 g, 10 mmol). The mixture was heated under reflux under nitrogen for 6 hours after cooling to room temperature, the reaction mixture was acidified with 2 M HCl aqueous solution to pH 4 and concentrated. The residual aqueous solution was extracted with ethyl acetate (3×150 mL); the organic portion was washed with brine (150 mL) and dried over anhydrous sodium sulfate. The solid obtained after evaporation of the solvent was purified through column chromatography.

### 2-Methyl-4-(5-methyl-2-furyl)methylene-1-[4-(benzylaminocarbonylmethyl)phenyl]-5-oxo-1H-imidazole (Compound (Id))

This compound was prepared according to the procedure described for the synthesis of compound (Id). Yield, 60%; mp 198-200° C.; 1H NMR (DMSO-d₆) ppm δ: 9.81 (d, 1H, NH), 8.57 (s, 1H, furan), 8.03 (s, 1H, furan), 7.95-7.20 (m, 9H, Ar—H), 6.96 (s, 1H, ArCH═), 4.21 (s, 2H, CH₂), 3.77 (s, 2H, CH₂), 2.44 (s, 3H, CH₃), 2.16 (s, 3H, CH₃); LC-MS (ESI), RT=2.3 min, m/z 416 [M+3].

### Example 2: Biological Screening Techniques

In Silico analysis of compound (Id) Molecular Targets and Enriched Pathways were performed as follows. The top 100 molecular targets of compound (Id) were deduced using the SwissTargetPrediction tool. The molecular targets of compound (Id) were used to identify the enriched pathways based on KEGG, Reactome, Panther, Wikipathway Cancer, and WikiPathways using the Over Representation Analysis (ORA) method with the WebGestalt tool.

### Example 3: Evaluation of Compound (Id) for Inhibition of Colorectal Carcinoma (CRC) Cell Lines Techniques

Cell culture of colorectal carcinoma cell lines was performed as follows. DLD-1, HCT116, and HT-29 cells were purchased from CLS Cell Line Service GmbH (Eppelheim, Germany) and cultured in Dulbecco's Modified Eagle Medium (DMEM) high glucose, GlutaMAX™ Supplement, pyruvate (Thermo Fisher Scientific, Paisley, UK) supplemented with 10% fetal bovine serum (FBS; Thermo Fisher Scientific), 200 U/mL penicillin, and 200 μg/ml streptomycin (Thermo Fisher Scientific), at 37° C. with 5% CO₂ in a humidified incubator.

The CellTiter®-Blue Cell Viability assay was acquired from Promega Corporation (Madison, WI, USA). Cell viability assay was performed as follows: DLD-1 (4×10³/well), HCT116 (4×10³/well), and HT-29 (7×10³/well) were incubated with a concentration gradient of compound (Id) ranging from 0.01 to 20 μM, in 96-well plates for 48 h at 37° C. Subsequently, 20 μl CellTiter®-Blue Cell Viability reagent was added to each well and incubated for an additional 2 h for the development of fluorescence. The fluorescence emission was measured at 590 nm using a SpectraMax® i3× Multi-Mode microplate reader (Molecular Devices, LLC, San Jose, CA, USA) and plotted against drug concentrations to determine the mean inhibitory concentration of compound (Id) producing 50% decrease in cell viability (IC₅₀). Experiments were performed in triplicates.

Apoptosis assay for colorectal carcinoma cell lines model was performed as follows. Cells were cultured in T25 culture flasks at a density of 3×105 cells for DLD-1, 4×105 cells for HCT116, and 5×105 cells for HT29 cells and treated with IC₅₀ and 2×IC₅₀ of compound (Id) for 48 h. The experiments were performed in triplicates. To measure apoptosis, an Annexin V APC staining assay (BD Biosciences, San Jose, CA, USA) was performed according to the manufacturer's recommendations, and 10,000 events were acquired using a FACSAria III flow cytometer (BD Biosciences, San Jose, CA, USA).

### Example 4: Evaluation of Compound (Id) for Inhibition of Leukemia Cell Lines Techniques

Cell culture of leukemia cell lines model was performed as follows. HL60, K562, and NB4 cell lines were purchased from CLS Cell Line Service GmbH (Eppelheim, Germany). MV4-11 cell line was purchased from American Type Culture Collection (ATCC; Virginia, USA). KG1α also referred to as KG1a was a gift from Prof. Ghulam Mufti (King College London, UK). Kasumi-1 cell line was purchased from Nawah Scientific (Cairo, Egypt). All cell lines were cultured in Roswell Park Memorial Institute-1640 (RPMI-1640) culture medium supplemented with 10% fetal bovine serum (FBS) (Thermo Fisher Scientific) and 0.1% ciprofloxacin (10 μg/ml; Cipla Limited; Mumbai, India). Cells were maintained in a humidified incubator at 37° C. with 5% CO₂.

Cell viability was determined using the CellTiter-Blue® Cell Viability Assay kit (Promega, Madison, WI, USA). Approximately 104 cells were plated in 96 well plate in the presence of increasing drug concentration in triplicate and incubated for 48 h at 37° C. in a humidified incubator. After the incubation period, 20 μL of the cell titer blue reagent was added to each well and the plate was incubated for additional 2 h for the development of florescence. Florescence was measured at 540Ex/590Em on SpectraMax i3 Multi-Mode Microplate Reader (Molecular Devices, LLC; San Jose, CA, USA). The results were plotted as % cell viability against the log concentration of the compound to determine its IC₅₀, which was obtained by non-linear regression model using GraphPad Prism 6.07 (GraphPad Software, Inc, USA).

Apoptosis assay for leukemia cells was performed as follows. Cells were cultured in 6-well plates at a density of 3× 105 cells per well and treated with approximately 2×IC₅₀ values of compound (Id) for 48 h. The experiments were performed in duplicates. To measure apoptosis, Annexin V APC and 7AAD staining (BD Biosciences, San Jose, CA, USA) was performed according to the manufacturer's recommendations, and 10,000 events were acquired using a FACSCanto flow cytometer (BD Biosciences, San Jose, CA, USA).

### Example 5: Maximum Kinetic Aqueous Solubility Techniques

Solubility assays were performed using Millipore MultiScreen® HTS-PFC Filter Plates designed for solubility assays (EMD Millipore, Billerica, MA). Assays were run in triplicate. The 96-well plates consist of two chambers separated by a filter. Liquid handling was performed using JANUS® Verispan and MTD workstations (Perkin Elmer, Waltham, MA). 4 μL of compound (Id) solution (10 mM in DMSO) was added to 196 μL of phosphate buffer (45 mM potassium phosphate, 45 mM sodium acetate, 45 mM ethanolamine, pH=7.4) in the top chamber to give a final DMSO concentration of 2% and a theoretical drug concentration of 200 μM. Plates were gently shaken for 90 min and then subjected to vacuum. Insoluble drug was captured on the filter. 160 μL of the filtrate was transferred to 96-well Griener UV Star® analysis plates (Sigma-Aldrich, St. Louis, MO) containing 40 μL of acetonitrile. The test compound concentration in the filtrates was measured by UV absorbance on a SpectraMax® Plus microplate reader (Molecular Devices, Sunnyvale, CA) using Softmax Pro software v.5.4.5. Absorbances at 5 wavelengths (280, 300, 320, 340, and 360 nm) were summed to generate the UV signal. Standard curves were generated by adding 4 μL of five concentrations of test compound in DMSO to 40 μL of acetonitrile in UV Star plates followed by 156 μL of the appropriate solubility medium. Analysis and statistics were performed using GraphPad® Prism v.5.04. Data were reported as the maximum concentration observed in the filtrate.

### Example 6: Stability in Mouse and Human Liver Microsomes Techniques

The clearance of compound (Id) in mouse or human liver microsomes was determined at 37° C. Both mouse and human liver microsomes were obtained from Merck KGaA, (Darmstadt, Germany), Cat. No. M9441 (Mouse liver microsomes) and M0317 (Human liver microsomes). Assays were conducted in triplicate in 96-deep well polypropylene plates. The compound was incubated with pooled liver microsomes from male CD-1 mice (Life Technologies, Grand Island, NY), tetra-sodium NADPH and magnesium chloride for 60 min at 37° C. with gentle shaking. At five time points, reaction mixture aliquots were transferred to 96-shallow well stop plates on ice containing acetonitrile with 0.1 μM propafenone. Control reactions (lacking NADPH) were performed in a similar manner to demonstrate NADPH dependency of compound loss and assess the potential for hydrolysis of the compound in liver tissue. Standard curves for test compounds were generated using 5 concentrations in triplicate that were processed as above but with zero incubation time. Stop plates were centrifuged at 2000 g for 10 min and then supernatant aliquots were transferred to a Waters Aquity® UPLC 700 μL 96-well sample plate with cap mat (Waters, Milford, MA). The amount of compound remaining in the supernatant was quantified by LC/MS/MS using a Waters Xevo TQ MS (electrospray positive mode) coupled to a Waters Aquity® UPLC (BEH column, C18). Propafenone was used as the internal standard. GraphPad® Prism v 5.04 was used for nonlinear fitting of time course data to generate t_{1/2} values.

The compound (Id) was assessed for its ability to inhibit the three major human CYP450 enzymes, 3A4, 2D6 and 2C₉. Assays were run in triplicate. Expressed enzymes in insect supersomes (Fisher Scientific, Waltham, MA) were used to minimize non-specific binding and membrane partitioning issues. The 3A4 assay uses testosterone as a substrate and analysis was performed by LC/MS/MS on a Waters Xevo TQ instrument as described above using positive electrospray ionization. Assay acceptance criteria was 20% for all standards and 25% for the LLOQ. The 2D6 and 2C₉ assays use fluorescent substrates (3-[2-(N,N-diethylamino)ethyl]-7-methoxy-4-methylcoumarin for 2D6; 7-methoxy-4-(trifluoromethyl) coumarin for 2C₉) and were analyzed on an Envision plate reader. GraphPad® Prism v 5.04 was used for nonlinear fitting of data to generate IC₅₀ values.

Stability in mouse plasma was tested as follows. The assay was conducted at 37° C. in triplicate. Test compound or procaine (positive control) was tested at a final concentration of 1 μM in either 2.5% DMSO/CD-1 mouse plasma (Innovative Research, Novi, MI; sodium heparin added as anticoagulant; pH adjusted to 7.4 with 2N HCl on day of use) or 2.5% DMSO/PBS (pH 7.4:136.9 mM NaCl, 2.68 mM KCl, 8.1 mM Na₂HPO₄, 1.47 mM KH₂PO₄, 0.9 mM CaCl₂, 0.49 mM MgCl₂). At seven time points, reaction mixture aliquots were transferred to 96-shallow well stop plates on ice containing acetonitrile with 0.1 μM propafenone. Samples were analyzed by LC/MS/MS on a Waters Xevo TQ instrument as described above using positive electrospray ionization. Assay acceptance criteria was 20% for all standards and 25% for the LLOQ. GraphPad® Prism v. 5.04 was used for nonlinear fitting of data to generate t_{1/2} values.

Binding to mouse plasma proteins was determined as follows. The equilibrium dialysis method for determining plasma protein binding was performed using 96-well dialyzer plates with molecular weight cutoff of 5K (Harvard Apparatus, Holliston, MA) and a dual-plate rotator set to maximum speed (Harvard Apparatus, Holliston, MA) located in a 37° C. incubator with a 10% CO₂-atmospheric environment. Assays were run in triplicate. The test compound was added to CD-1 mouse plasma (Innovative Research, Novi, MI, sodium heparin added as anticoagulant; pH adjusted to 7.4 with 2N HCl on day of use) in DMSO (final DMSO concentration 0.4%) to give 10 μM final concentration. Drug/plasma mixture and buffer (Dulbecco's phosphate-buffered saline 1× without calcium and magnesium, Mediatech, Inc., Herndon, VA) were placed in their respective sides, wells were capped, and the plate was placed in the rotator and allowed to dialyze for 22 h. Following dialysis, aliquots of buffer and plasma mixture were removed and mixed with aliquots of the opposite matrix in 96-well deep plates. Concentrations of analyte from each side of the dialysis plate were determined by LC/MS/MS on a Waters Xevo TQ instrument as described above using positive electrospray ionization. Assay acceptance criteria was 20% for all standards and 25% for the LLOQ. The fraction unbound was calculated by dividing the drug concentration in the buffer side of the dialysis plate by the drug concentration in the plasma side.

### Example 7: Biological Screening Results

To understand the role of compound (Id) as an anticancer compound, the SwissTargetPrediction tool was used to decode the molecular targets of compound (Id). The compound (Id) target molecules were then used to identify enriched pathways, gene ontologies, and kinase targets using the WebGestalt tool as previously described. The enrichment results for the top pathways affected by compound (Id) treatment are summarized in Table 1.

TABLE 1
Enrichment results for the top pathways affected by Compound (Id) treatment.

| Gene Set | Description | Size | Expect | Ratio | P Value | FDR |
|---|---|---|---|---|---|---|
| hsa01521 | EGFR tyrosine | 79 | 0.97309 | 13.360 | 8.56E−12 | 6.98E−10 |
| | kinase inhibitor | | | | | |
| | resistance | | | | | |
| hsa05200 | Pathways in cancer | 526 | 6.4790 | 4.0129 | 4.19E−10 | 1.71E−08 |
| hsa04066 | HIF-1 signaling | 100 | 1.2318 | 8.9303 | 3.11E−08 | 7.24E−07 |
| | pathway | | | | | |
| hsa04014 | Ras signaling | 232 | 2.8577 | 5.2490 | 1.20E−07 | 2.45E−06 |
| | pathway | | | | | |
| hsa04151 | PI3K-Akt signaling | 354 | 4.3604 | 3.8987 | 1.1348E−06 | 1.61E−05 |
| | pathway | | | | | |
| hsa04152 | AMPK signaling | 120 | 1.4781 | 6.7654 | 1.8695E−06 | 2.26E−05 |
| | pathway | | | | | |
| hsa04140 | Autophagy | 128 | 1.5767 | 6.3426 | 3.3708E−06 | 3.79E−05 |
| hsa05210 | Colorectal cancer | 86 | 1.0593 | 7.5521 | 9.4218E−06 | 8.53E−05 |
| hsa04210 | Apoptosis | 136 | 1.6752 | 5.3725 | 4.0793E−05 | 2.83E−04 |

### Example 8: Inhibition of Colorectal Carcinoma Cell Lines Results

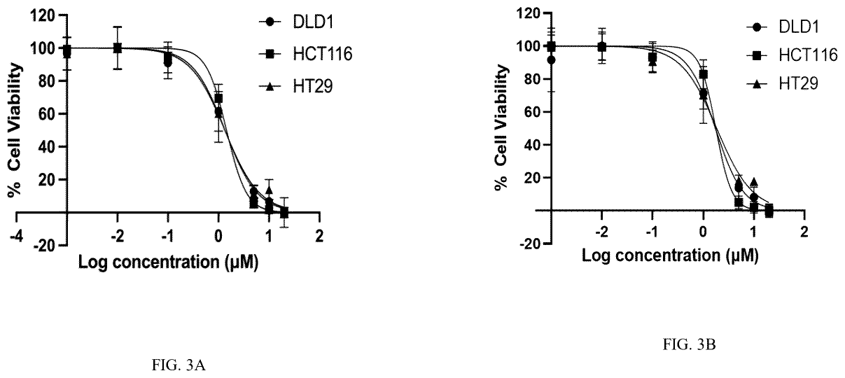

To confirm the activity of compound (Id) cell viability experiments on different colorectal carcinoma cell lines DLD1, HCT116, and HT29 in the absence or presence of a compound (Id) concentration gradient were performed. The cytotoxic activity was tested in parallel to KIM-161 in the same assays for comparison. Compound (Id) demonstrated IC₅₀ values of 1.35 μM, 1.46 μM, and 1.34 μM for DLD1, HCT116, and HT29 cells, respectively (Table 2 and FIG. 3A). The cytotoxic effect of compound (Id) on the cell lines was comparable to that of KIM-161 (Table 2 and FIG. 3B).

TABLE 2
Cytotoxic activities of Compound (Id) against
three colorectal cancer cell lines.

| | DLD1 | HCT116 | HT29 |
|---|---|---|---|
| Compound | IC₅₀ (μM)* | IC₅₀ (μM) | IC₅₀ (μM) |
| Compound (Id) | 1.350 | 1.458 | 1.338 |
| KIM-161 | 1.730 | 1.767 | 1.842 |

*IC₅₀. Concentration of the compound at which viability of cells are 50% of the control (untreated cells). Data are mean of triplicates experiments. Calculations were made using GraphPad Prism version 8.0.1

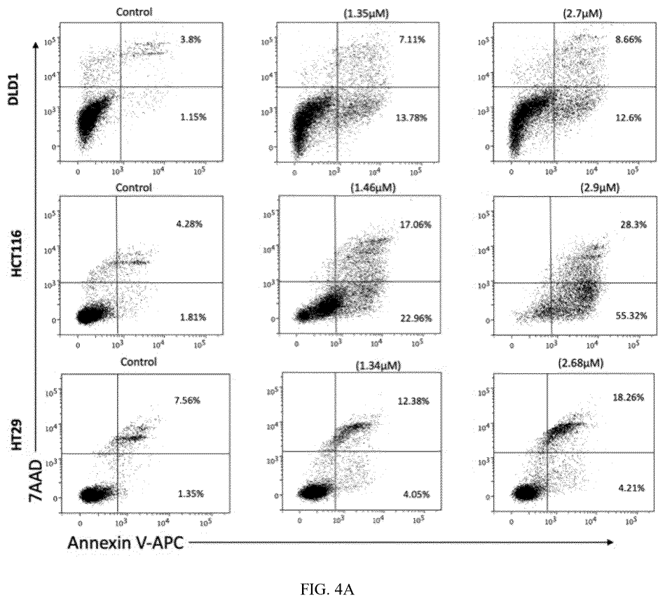

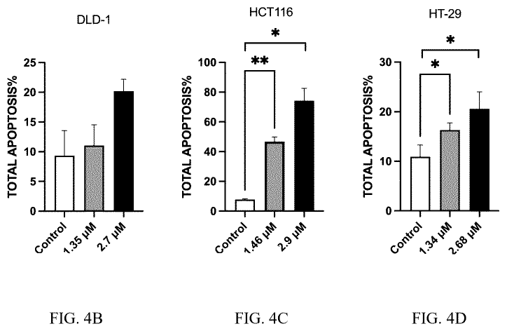

To understand the mechanism of compound (Id), an apoptosis assay was performed using flow cytometry. Compound (Id) treatment led to increased apoptosis compared to basal conditions in DLD1, HCT116, and HT29 cells (FIGS. 4A-4D) at its IC₅₀ and 2×IC₅₀ concentrations in a dose dependent manner. These findings indicate that compound (Id) is a potential inhibitor of CRC growth and proliferation through induction of apoptosis.

### Example 9: Inhibition of Leukemia Cell Lines Results

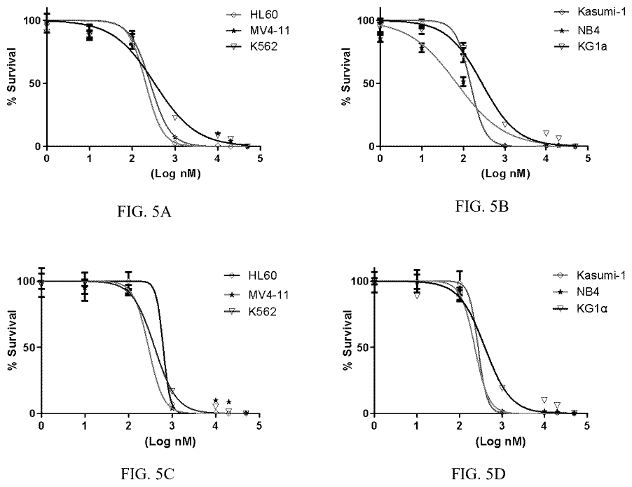

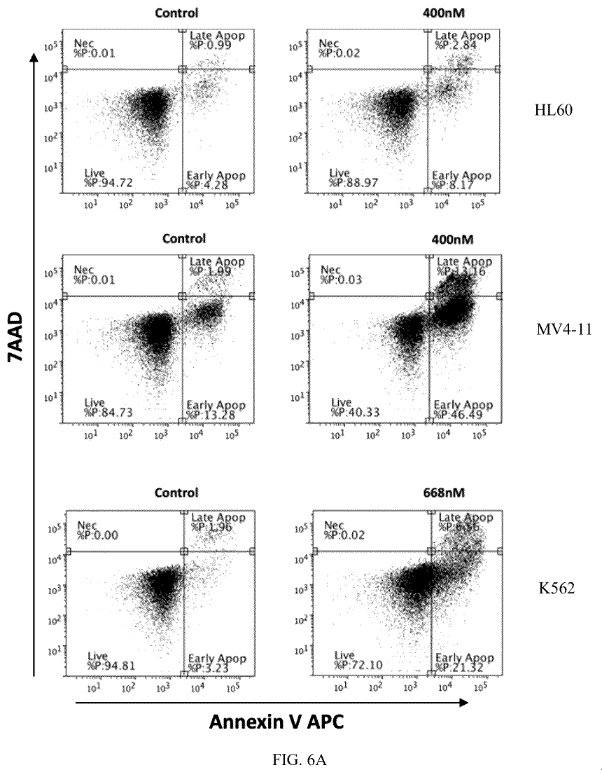

The cytotoxic activity of compound (Id) on HL60, MV4-11, K562, Kasumi-1, NB4, and KG1α cell lines was tested. The cytotoxic activity was tested in parallel to KIM-161 in the same assays for comparison. The results presented in FIGS. 5A-5D and Table 3 illustrate potent inhibition of cell growth with IC₅₀ values at the sub-micromolar range, which is comparable to KIM-161. Compound (Id) demonstrated the following IC₅₀ values: HL60 (0.443 μM), MV4-11 (0.254 μM), K562 (0.437 μM), Kasumi-1 (0.221 μM), NB4 (0.189 μM), and KG1α (0.488 μM). Myeloid leukemia cell lines HL60, MV4-11, K562, Kasumi-1, NB4, and KG1α cells, demonstrated higher sensitivity to compound (Id) treatment with sub micromolar IC₅₀ values as compared to CRC cell line model (Tables 2 and 3).

TABLE 3
Cytotoxic activities of Compound (Id)
against six leukemia cell lines.

| Compound | HL60 | MV4-11 | K562 | Kasumi-1 | NB4 | KG1a |
|---|---|---|---|---|---|---|
| KIM-161 | 0.238 | 0.361 | 0.282 | 0.166 | 0.148 | 0.283 |
| (IC₅₀, μM) | | | | | | |
| Compound (Id) | 0.443 | 0.254 | 0.437 | 0.221 | 0.189 | 0.488 |
| (IC₅₀, μM) | | | | | | |

*IC₅₀. Concentration of the compound at which viability of cells are 50% of the control (untreated cells). Data are mean of triplicate experiments. Calculations were made using GraphPad Prism version 8.0.1

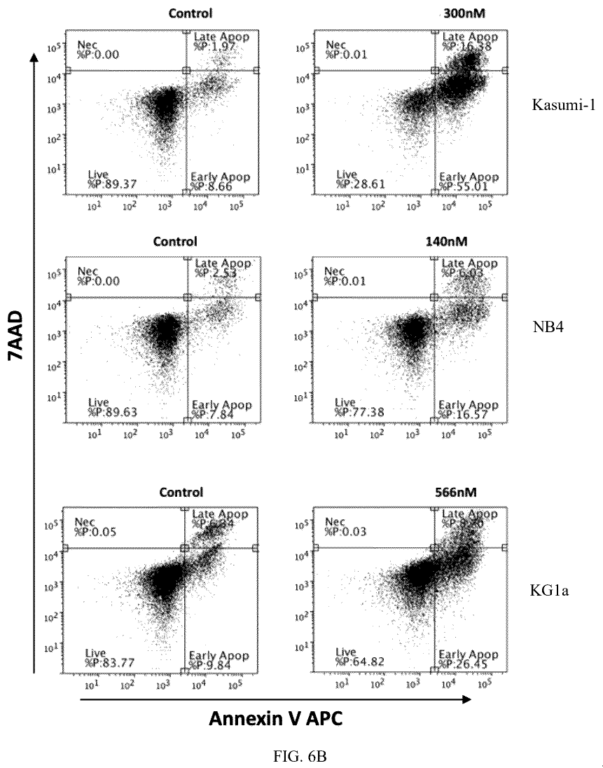

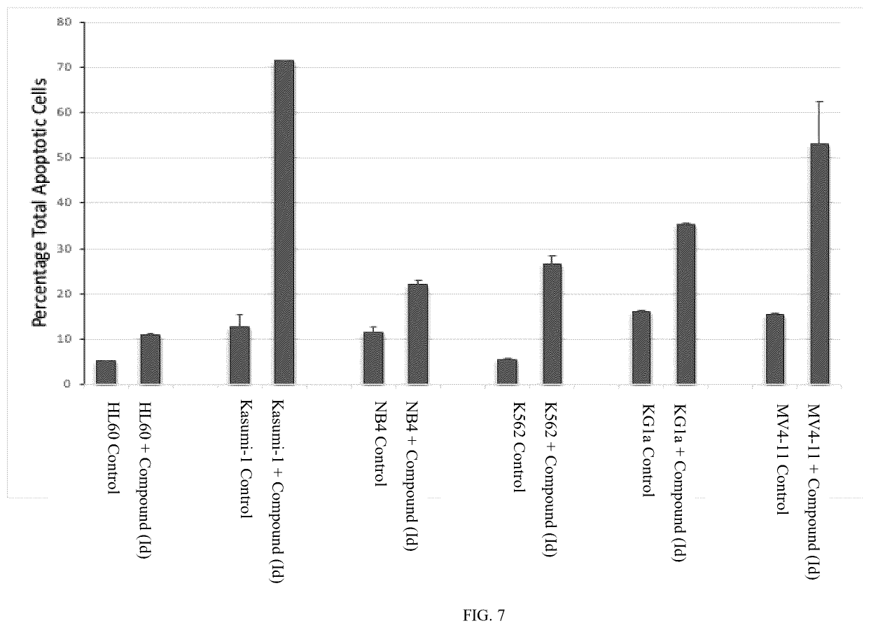

The induction of apoptosis in the six myeloid leukemia cell lines was evaluated. Treatment of HL60, MV4-11, K562, Kasumi-1, NB4, and KG1α cell lines with compound (Id) for 48 h followed by flow cytometry based analysis of surface Annexin V expression and 7AAD staining revealed strong apoptosis at the mentioned doses (FIGS. 6A-6B and 7). Particularly notable was the effect of compound (Id) on Kasumi-1 cells which showed very strong induction of apoptosis. This is due to the fact that the Kasumi-1 cells carry a c-kit mutation that confer constitutive ligand-independent activation of the KIT receptor, leading to dependence of Kasumi-1 cells on Ras and PI3K-Akt signaling pathways. As shown in Table 1, the enrichment results indicate compound (Id) inhibits kinases of the Ras and PI3K-AKT signaling pathways. The data indicate that compound (Id) is effective in killing leukemia cells.

### Example 10: Physicochemical Profiling of Compound (Id) Techniques and Results

Profiling experiments of compound (Id) were performed in-silico and in vitro. For the in-silico testing, the calculated log P (c Log P) and topological polar surface area (TPSA) were determined using Marvin calculator plugin in Marvin Sketch from Chemaxon (Budapest, Hungary). The log P calculator utilizes a variation on the atomic fragment method, while the TPSA calculator employs the fragment summation method. The compound (Id) has a c Log P value of 2.47, and the TPSA was 75.2 (Table 4).

A battery of high-throughput in vitro assays was performed to evaluate ADME properties of compound (Id). The results were obtained as follows (Table 4): maximum aqueous solubility in 2% DMSO/PBS 79.8 μM; MLM stability 16.6 min; HLM stability 30 min; % compound (Id) remaining in MLM and HLM in the presence and absence of NADPH did not change after 60 min. The compound was stable in mouse plasma at 37° C. for over 300 min, and the free fraction of compound (Id) after incubating with mouse plasma proteins was 3.2%. Additionally, no inhibition in the activity of CYP450 3A4, 2C9, and 2D6 was observed by compound (Id) (IC₅₀>10,000 μM).

The values of the parameters/properties c Log P (2.47), PTSA (75.2) and maximum kinetic aqueous solubility (79.8 μM) are indications of an improved aqueous solubility for compound (Id) as a result of introducing the furan ring as the 5-arylidene moiety, as compared to the values for KIM-161 (c Log P 3.39, TPSA 71, Max. aq. Sol. 19.9 μM). The improved properties of compound (Id) facilitate preparing the compound in a variety of pharmaceutical preparations for oral, intravenous (IV), and/or subcutaneous (SC) administration of the in vivo studies and eventually, clinical trials. Collectively, these findings indicate that compound (Id) is a promising compound that prevents caner growth and proliferation of malignant diseases.

TABLE 4
In vitro Physicochemical/ADME Data for Compound (Id).

| Comparison | KIM-161 | Compound (Id) |
|---|---|---|
| cLogP | 3.39 | 2.47 |
| TPSA | 71 | 75.2 |
| Max. aq. Sol. (μM) | 19.9 | 79.8 |
| Stability MLM¹ t_{1/2} (min)² | 14.8 | 16.6 |
| Stability HLM¹ t_{1/2} (min)² | 22.9 | 30.3 |
| Mic. Ctrl. Mouse % @60 min³ | 112 | 90 |
| Mic. Ctrl. Human % @60 min³ | 113 | 118 |
| CYP3A4 IC₅₀ (μM) | >10,000 | >10,000 |
| CYP2D6 IC₅₀ (μM) | >10,000 | >10,000 |
| CYP2C9 IC₅₀ (μM) | >10,000 | >10,000 |
| Mouse Plasma Stability t_{1/2} (min) | >300 | >300 |
| Mouse PPB (% free) | 5 | 3.2 |

¹HLM = Human Liver Microsomes.
²Assay carried out in presence of NADPH.
³Assay carried out without NADPH; percent remaining after 60 min incubation.

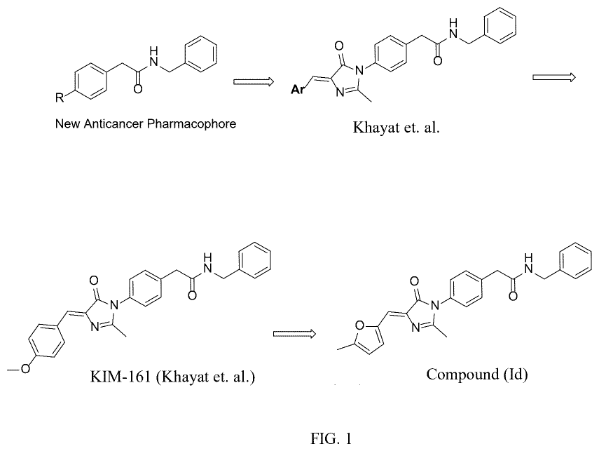

Compound (Id) was designed as a derivative having a 5-methylfuryl moiety as the 5-arylidene group as a replacement for the 4-methoxyphenyl found in KIM-161. The purpose of this modification was to increase the aqueous solubility of the compound while maintaining therapeutic benefit against malignant tumors, exemplified by colorectal carcinoma and leukemia cell lines (FIG. 1). Unexpectedly, not only is the solubility improved but also the effectiveness in treating some colorectal cancer and leukemia is improved as shown in Table 5.

TABLE 5
Activities of KIM-161 and Compound (Id).

| | IC₅₀ (μM) HT29 | IC₅₀ (μM) | Max. aq. |
|---|---|---|---|
| Compound | (Colorectal cancer) | MV4-11 (Leukemia) | Sol. (μM) |
| KIM-161 | 1.842 | 0.361 | 19.9 |
| Compound (Id) | 1.338 | 0.254 | 79.8 |

1: A compound, having a following general formula (I): 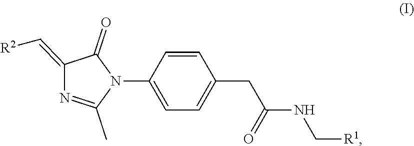
a salt thereof, a solvate thereof, a tautomer thereof, a stereoisomer thereof, or a mixture thereof;
wherein:
R¹ is selected from the group consisting of an optionally substituted alkyl, an optionally substituted cycloalkyl, an optionally substituted arylalkyl, and an optionally substituted aryl, and
R² is an optionally substituted heterocycle.

2: The compound of claim 1, wherein R¹ is an optionally substituted aryl.

3: The compound of claim 1, wherein R¹ is a phenyl.

4: The compound of claim 1, wherein R² is an optionally substituted heteroaryl.

5: The compound of claim 1, wherein R² is an optionally substituted heteroaryl having a 5-membered ring.

6: The compound of claim 1, wherein R² is an alkyl substituted furyl, imidazolyl, thiazolyl, thienyl, oxazolyl, isoxazolyl, triazolyl, pyrazolyl, isothiazolyl, or pyrroyl.

7: The compound of claim 1, wherein R² is an optionally substituted heteroaryl having a 6-membered ring.

8: The compound of claim 1, wherein R² is an alkyl substituted pyridyl, pyrimidinyl, pyrazinyl, pyridazinyl, or triazinyl.

9: The compound of claim 1, wherein the compound has the following formula (Id):

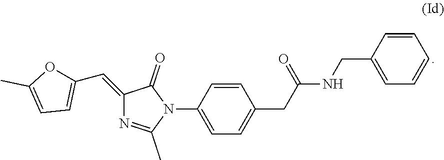 [SMILES: CC1=N/C(=C\c2ccc(C)o2)C(=O)N1c1ccc(CC(=O)NCc2ccccc2)cc1]

10: The compound of claim 1, having a maximum kinetic aqueous solubility of greater than 75 μM.

11: A pharmaceutical composition, comprising:
the compound of claim 1; and
a pharmaceutically acceptable carrier and/or excipient.

12: The pharmaceutical composition of claim 11, comprising 0.1-10 wt. % of the compound relative to a total weight of the pharmaceutical composition.

13: The pharmaceutical composition of claim 11, wherein the pharmaceutically acceptable carrier and/or excipient is at least one selected from the group consisting of a buffer, an inorganic salt, a fatty acid, a vegetable oil, a synthetic fatty ester, a surfactant, and a polymer.

14: The pharmaceutical composition of claim 11, wherein the compound has the following formula (Id):

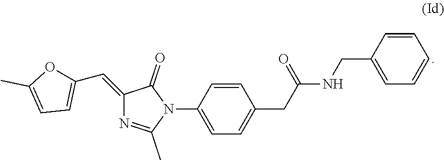 [SMILES: CC1=N/C(=C\c2ccc(C)o2)C(=O)N1c1ccc(CC(=O)NCc2ccccc2)cc1]

15: A method for treating cancer in a subject, comprising administering to the subject a therapeutically effective amount of the compound of claim 1.

16: The method of claim 15, wherein the cancer is colon cancer or leukemia.

17: A method for decreasing an amount of colorectal cancer cells, comprising contacting the compound of claim 1 with the colorectal cancer cells.

18: The method of claim 17, wherein the compound has an IC₅₀ value of 1-2 μM for the colorectal cancer cells.

19: A method for decreasing an amount of leukemia cancer cells, comprising contacting the compound of claim 1 with the leukemia cancer cells.

20: The method of claim 19, wherein the compound has an IC₅₀ value of 0.1-1 μM for the leukemia cancer cells.
